(19) United States
(12) Patent Application Publication
(10) Pub. No.: US 2024/0391885 A1
(43) Pub. Date: Nov. 28, 2024

(54) FORMS OF LINERIXIBAT

(71) Applicant: GLAXOSMITHKLINE INTELLECTUAL PROPERTY (NO. 2) LIMITED, Stevenage (GB)

(72) Inventor: Stephen CARINO, Collegeville, PA (US)

(21) Appl. No.: 18/258,572

(22) PCT Filed: Dec. 21, 2021
(86) PCT No.: PCT/EP2021/086929

§ 371 Date: Jun. 21, 2023

Related U.S. Application Data
(60) Provisional application No. 63/129,676, filed on Dec. 23, 2020.

(51) Int. Cl. C07D 281/10; A61K 9/20; A61K 9/28; A61K 31/554

(52) U.S. Cl. CPC C07D 281/10; A61K 9/2013; A61K 9/2054; A61K 9/2853; A61K 31/554

(57) ABSTRACT

Disclosed are crystalline and amorphous forms of linerixibat and pharmaceutical compositions containing the same. Also disclosed are processes for the preparation thereof and methods for use thereof. Also disclosed is solubility and dissolution information for linerixibat.

### FIELD OF THE INVENTION

The present invention relates to crystalline and amorphous forms of linerixibat and solubility and dissolution profiles of linerixibat. Linerixibat has the structure of Formula (I).

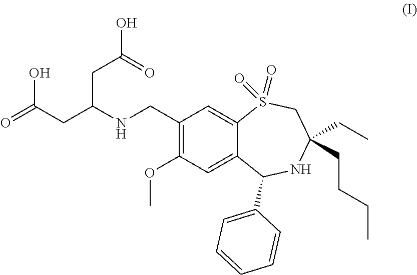 [SMILES: CCCC[C@]1(CC)CS(=O)(=O)c2cc(CNC(CC(=O)O)CC(=O)O)c(OC)cc2[C@@H](c2ccccc2)N1]

The present invention provides crystalline forms of linerixibat, such as Form I, Form II, Form III, Form IV, Form V of linerixibat, or amorphous linerixibat, and a composition comprising Form I, Form II, Form III, Form IV, Form V or amorphous linerixibat or a mixture of two or more thereof. The present invention also provides methods for making the crystalline forms of linerixibat, a pharmaceutical composition comprising the crystalline forms of linerixibat, and methods of treating cholestatic pruritus in patients with primary biliary cholangitis (PBC) using the crystalline forms of linerixibat.

### BACKGROUND OF THE INVENTION

3-({[(3R,5R)-3-butyl-3-ethyl-7-(methyloxy)-1,1-dioxido-5-phenyl-2,3,4,5-tetrahydro-1,4-benzothiazepin-8-yl]methyl}amino)pentanedioic acid, also known as linerixibat, GSK2330672, GSK2330672B and sometimes abbreviated as GSK672 (hereinafter “linerixibat”) is a selective inhibitor of the human ileal bile acid transporter (IBAT) and is in clinical trials for treatment of cholestatic pruritus in patients with Primary Biliary Cholangitis (PBC).

International Patent Application Publication No. WO2011/137135 describes preparation of a series of compounds, including linerixibat in Example 26. Methods for synthesizing linerixibat are also described in WO2016/020785 and WO2018/002827. These three International Patent Application Publications are incorporated herein by reference in their entirety.

### SUMMARY OF THE INVENTION

In a first aspect of the invention, there is provided a crystalline form of linerixibat, which is Form III.

In a second aspect of the invention, there is provided a mixture of i) crystalline Form III of linerixibat and ii) crystalline Form I of linerixibat.

In a third aspect of the invention, there is provided a crystalline form of linerixibat, which is Form II.

In a fourth aspect of the invention, there is provided a crystalline form of linerixibat, which is Form IV.

In a fifth aspect of the invention, there is provided a crystalline form of linerixibat, which is Form V.

In a sixth aspect of the invention, there is provided an amorphous form of linerixibat.

In a seventh aspect of the invention, there is provided a composition comprising linerixibat in a form disclosed herein.

In an eighth aspect of the invention, there is provided a pharmaceutical composition comprising the composition of linerixibat in a form disclosed herein, and a pharmaceutically acceptable excipient.

In a ninth aspect of the invention, there is provided a method of treating cholestatic pruritus in a patient with primary biliary cholangitis comprising administering to the patient an effective amount of the pharmaceutical composition disclosed herein.

In a tenth aspect of the invention, there is provided an oral dosage form of linerixibat, characterised in that linerixibat is in a form which has a solubility of ≥0.4 mg/mL at an intestinal pH of about 6.8 and wherein dissolution of the oral dosage form is complete in ≤1 hour.

In an eleventh aspect of the invention, there is provided an oral dosage form of linerixibat which exhibits a dissolution profile substantially in accordance with FIG. 24.

In a twelfth aspect of the invention, there is provided an IBAT inhibitor which exhibits a solubility profile 80-125% equivalent to that shown in FIG. 25.

In a thirteenth aspect of the invention, there is provided an IBAT inhibitor which exhibits a solubility profile substantially in accordance with FIG. 25.

### BRIEF DESCRIPTION OF THE DRAWINGS

FIG. 1 shows an X-ray powder diffraction pattern of Form I of linerixibat.

FIG. 2 shows a Differential Scanning Calorimetry (DSC) trace of Form I of linerixibat.

FIG. 3 shows a ¹³C solid-state NMR (SSNMR) spectrum of Form I of linerixibat.

FIG. 4 shows an X-ray powder diffraction pattern of Form III of linerixibat.

FIG. 5 shows a Differential Scanning Calorimetry (DSC) trace of Form III of linerixibat.

FIG. 6 shows a ¹³C SSNMR spectrum of Form III of linerixibat.

FIG. 7 shows an overlay of X-ray powder diffraction patterns of Form I and Form III of linerixibat.

FIG. 8 shows a ¹³C SSNMR spectral overlay of Form I (bottom) and Form III (top).

FIG. 9 shows an expanded spectral region of ¹³C SSNMR as an example of characteristic peaks of Form I and Form III.

FIG. 10 shows an overlay of X-ray powder diffraction patterns of Form I, Form III, and a sample of compressed Form I.

FIG. 11 shows a ¹³C SSNMR spectrum of a sample of compressed Form I, and an expanded section of the same spectrum, which indicates the presence of both Form I and Form III.

FIG. 12 shows an X-ray powder diffraction pattern of Form II of linerixibat.

FIG. 13 shows a Differential Scanning Calorimetry (DSC) trace of Form II of linerixibat.

FIG. 14 shows a ¹³C solid-state NMR (SSNMR) spectrum of Form II of linerixibat.

FIG. 15 shows an X-ray powder diffraction pattern of Form IV of linerixibat.

FIG. 16 shows a Differential Scanning Calorimetry trace of Form IV of linerixibat.

FIG. 17 shows a ¹³C solid-state NMR (SSNMR) spectrum of Form IV of linerixibat

FIG. 18 shows an X-ray powder diffraction pattern of Form V of linerixibat.

FIG. 19 shows a Differential Scanning Calorimetry (DSC) trace of Form V of linerixibat.

FIG. 20 shows a ¹³C solid-state NMR (SSNMR) spectrum of Form V of linerixibat.

FIG. 21 shows an overlay of X-ray powder diffraction patterns of Form I, Form II, Form III, Form IV and Form V.

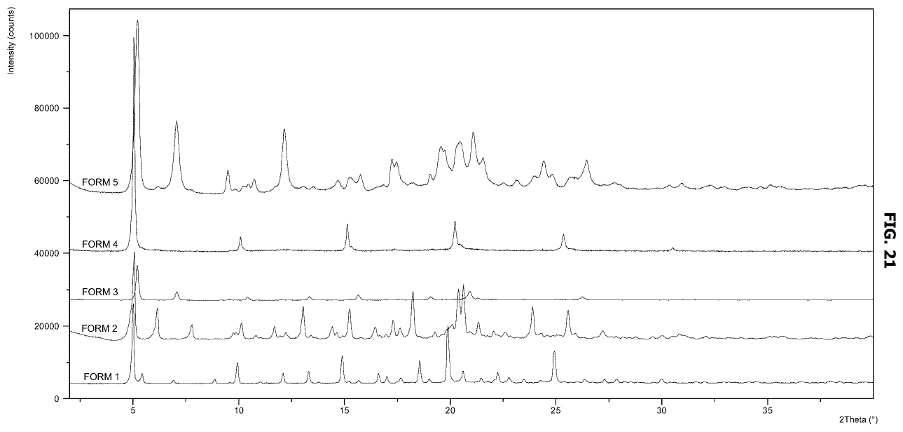

FIG. 22 shows a ¹³C SSNMR spectral overlay of Form I (top), Form II (middle) and Form III (bottom).

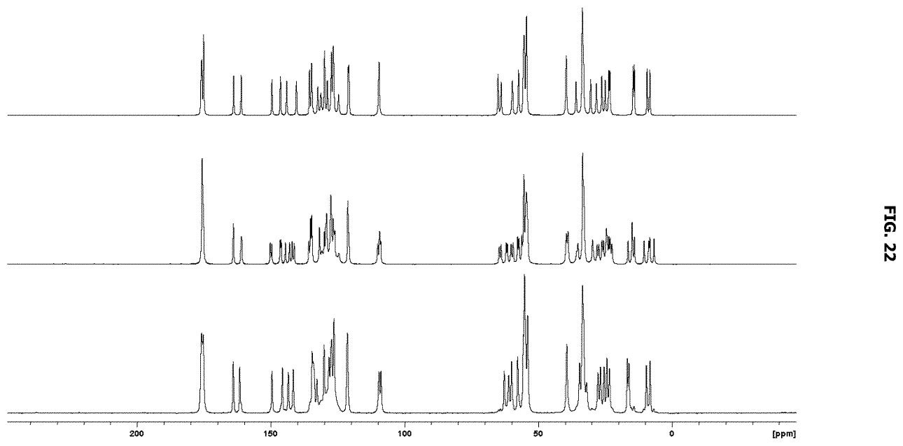

FIG. 23 shows a ¹³C SSNMR spectral overlay, from top to bottom: Form I, Form II, Form III, Form IV and Form V.

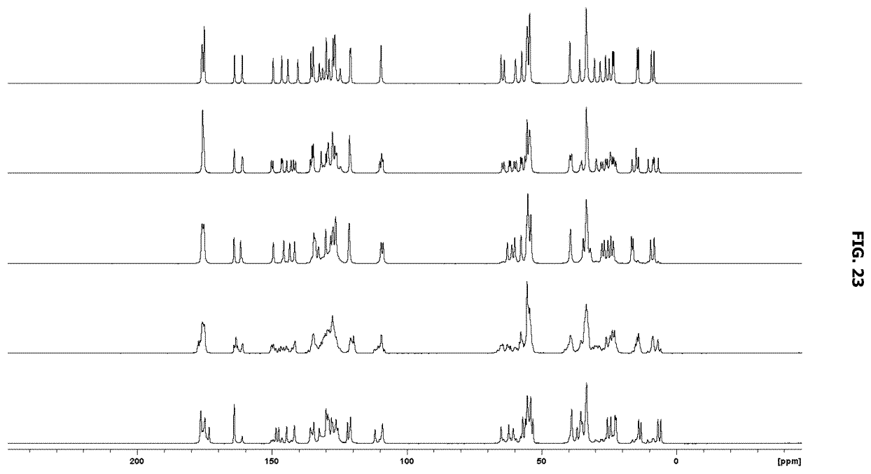

FIG. 24 shows a Dissolution Profile at pH 6.8 of linerixibat tablets, at about 37° C., 40 mg batches.

FIG. 25 shows a Solubility Profile for linerixibat drug substance Form I and Form III in biorelevant media, at 4 hrs, at about 37° C.

FIG. 26 shows a Solubility Comparison of Form I and Form III, at a range of between pH3 and pH5, at 4 h

### DETAILED DESCRIPTION OF THE INVENTION

The present invention is directed to crystalline forms of linerixibat.

Crystalline Form III of linerixibat (also referred to as “Form III” or “Form 3” herein) was discovered from a polymorph screen. Form III was also observed when Form I was subjected to mechanical stress and/or compaction by partial conversion of Form I to Form III.

Crystalline Form I of linerixibat (also referred to as “Form I” or “Form 1” herein) was the predominant form observed from a polymorph form screening, indicating that it is likely to be the most thermodynamically stable form at or around room temperature.

Crystalline Form II of linerixibat (also referred to as “Form II” or “Form 2” herein) was also discovered from a polymorph screen.

Crystalline Form IV of linerixibat (also referred to as “Form IV” or “Form 4” herein) was also discovered from a polymorph screen.

Crystalline Form V of linerixibat (also referred to as “Form V” or “Form 5” herein) was also discovered from a polymorph screen.

As used herein, when the term “about” is before a list of numbers, the term applies to each of the listed numbers.

### Form I of Linerixibat

As described in Example 26 of WO2011/137135, Form I of linerixibat can be prepared by crystallization from a mixture solvent of acetic acid and water.

Form I of linerixibat, a non-solvated crystalline form that melts with decomposition at onset temperature about 206° C. and peak temperature about 209° C., was identified as the predominant form of linerixibat from a polymorph screen study. Amorphous linerixibat and Form I were used as input material for polymorph screen experiments. Form I appears to be the most stable form relative to the other forms identified from the form screen. Form I was obtained from a variety of screening samples, and the solvents used include water, methanol, ethanol, acetone, acetonitrile and ethyl acetate.

In one embodiment, Form I is characterized by an X-ray powder diffraction (XRPD) pattern comprising at least three, at least four, at least five, at least six, at least seven, at least eight, or at least nine diffraction angles, when measured using Cu K_{α} radiation, selected from the group consisting of about 5.0, 5.5, 9.9, 12.1, 13.3, 14.9, 18.6, 19.9, 20.6, and 22.3 degrees 2θ.

In one embodiment, Form I is characterized by an X-ray powder diffraction (XRPD) pattern comprising at least three, at least four, at least five, at least six, at least seven, at least eight, or at least nine diffraction angles, when measured using Cu K_{α} radiation, selected from the group consisting of about 5.0, 5.5, 7.0, 8.9, 9.9, 12.1, 13.3, 14.9, 18.6, 19.9, 20.6, and 22.3 degrees 2θ. In one embodiment, Form I is characterized by an X-ray powder diffraction (XRPD) pattern comprising at least three, at least four, at least five, at least six, at least seven, at least eight, or at least nine diffraction angles (d-spacing), when measured using Cu K_{α} radiation, selected from the group consisting of about 5.0 (17.5), 5.5 (16.2), 7.0 (12.7), 8.9 (10.0), 9.9 (8.9), 12.1 (7.3), 13.3 (6.6), 14.9 (6.0), 18.6 (4.8), 19.9 (4.5), 20.6 (4.3), and 22.3 (4.0) degrees 2θ. In one embodiment, Form I is characterized by an XRPD pattern comprising at least three or at least four diffraction angles, when measured using Cu Kα radiation, selected from the group consisting of about 5.0, 5.5, 9.9, 12.1, 13.3, 14.9, 18.6, 19.9, 20.6, and 22.3 degrees 2θ.

In one embodiment, Form I is characterized by an XRPD pattern comprising at least four diffraction angles, when measured using Cu K_{α} radiation, selected from the group consisting of about 5.0, 5.5, 9.9, 14.9, 18.6, and 19.9 degrees 2θ. In one embodiment, Form I is characterized by an XRPD pattern comprising at least four diffraction angles (d-spacing), when measured using Cu K_{α} radiation, selected from the group consisting of about 5.0, 5.5, 7.0, 8.9, 9.9, 14.9, 18.6, and 19.9 degrees 2θ. In one embodiment, Form I is characterized by an XRPD pattern comprising at least four diffraction angles (d-spacing), when measured using Cu K_{α} radiation, selected from the group consisting of about 5.0 (17.5), 5.5 (16.2), 7.0 (12.7), 8.9 (10.0), 9.9 (8.9), 14.9 (6.0), 18.6 (4.8), and 19.9 (4.5) degrees 2θ.

In one embodiment, Form I is characterized by an XRPD pattern comprising at least three diffraction angles, when measured using Cu K_{α} radiation, selected from the group consisting of about 5.0, 5.5, 7.0, 8.9, 9.9, 14.9, 18.6, and 19.9 degrees 2θ. In one embodiment, Form I is characterized by an XRPD pattern comprising at least three diffraction angles (d-spacing), when measured using Cu K_{α} radiation, selected from the group consisting of about 5.0 (17.5), 5.5 (16.2), 7.0 (12.7), 8.9 (10.0), 9.9 (8.9), 14.9 (6.0), 18.6 (4.8), and 19.9 (4.5) degrees 2θ.

In one embodiment, Form I is characterized by an XRPD pattern comprising at least four diffraction angles, when measured using Cu K_{α} radiation, at about 5.0, 9.9, 14.9, 18.6, and 19.9 degrees 2θ. In one embodiment, Form I is characterized by an XRPD pattern comprising at least four diffraction angles, when measured using Cu K_{α} radiation, selected from the group consisting of about 5.0, 7.0, 8.9, 9.9, 14.9, 18.6, and 19.9 degrees 2θ. In one embodiment, Form I is characterized by an XRPD pattern comprising at least four diffraction angles (d-spacing), when measured using Cu K_{α} radiation, selected from the group consisting of about 5.0 (17.5), 7.0 (12.7), 8.9 (10.0), 9.9 (8.9), 14.9 (6.0), 18.6 (4.8), and 19.9 (4.5) degrees 2θ.

In one embodiment, Form I is characterized by an XRPD pattern comprising at least three diffraction angles, when measured using Cu K_{α} radiation, at about 9.9, 14.9, 18.6, and 19.9 degrees 2θ. In one embodiment, Form I is characterized by an XRPD pattern comprising at least three diffraction angles, when measured using Cu K_{α} radiation, selected from the group consisting of about 9.9, 14.9, 18.6, and 19.9 degrees 2θ. In one embodiment, Form I is characterized by an XRPD pattern comprising at least three diffraction angles (d-spacing), when measured using Cu K_{α} radiation, selected from the group consisting of about 9.9 (8.9), 14.9 (6.0), 18.6 (4.8), and 19.9 (4.5) degrees 2θ.

In one embodiment, Form I is characterized by an XRPD pattern comprising at least three diffraction angles, when measured using Cu K_{α} radiation, selected from the group consisting of about 5.0, 5.5, 7.0 and 8.9 degrees 2θ. In one embodiment, Form I is characterized by an XRPD pattern comprising at least three diffraction angles, when measured using Cu K_{α} radiation, selected from the group consisting of about 5.0 (17.5), 5.5(16.2), 7.0 (12.7), and 8.9(10.0) degrees 2θ.

In one embodiment, Form I is characterized by an X-ray powder diffraction (XRPD) pattern comprising at least three, at least four, at least five, at least six, at least seven, at least eight, or at least nine diffraction angles, when measured using Cu K_{α} radiation, selected from the diffraction angles shown in Table 1.

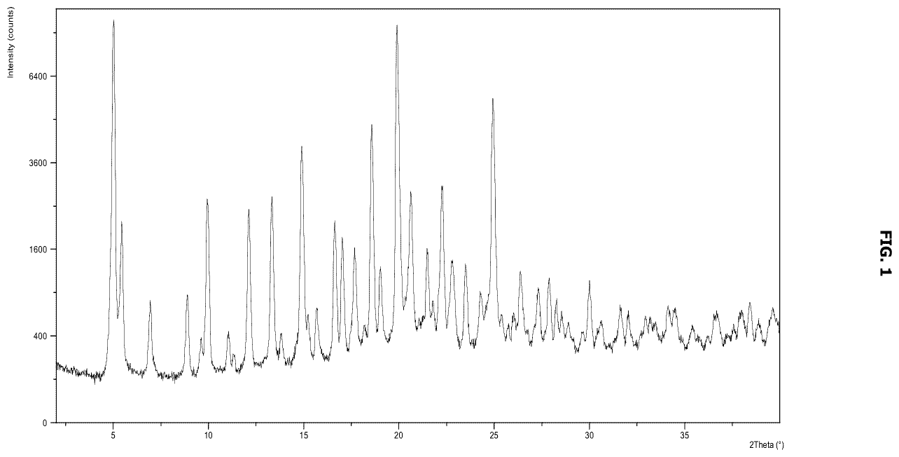

In one embodiment, Form I is characterized by an XRPD pattern substantially in accordance with FIG. 1.

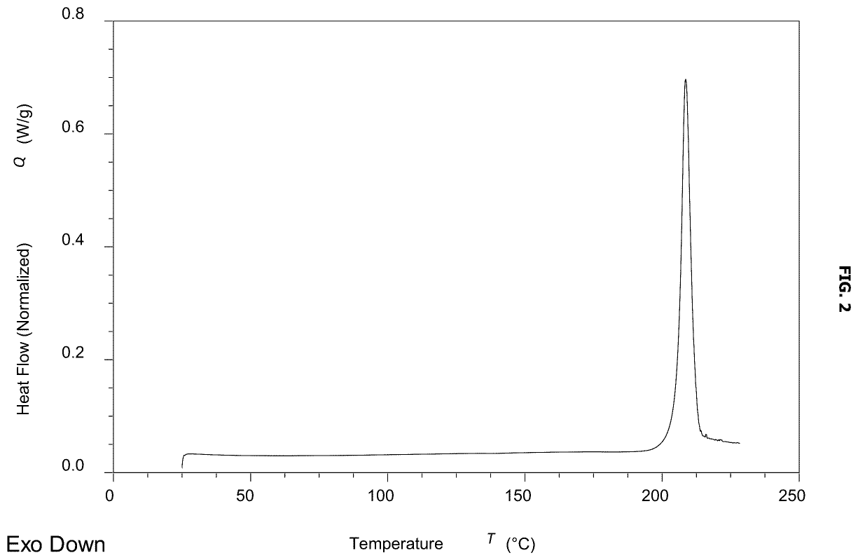

In one embodiment, Form I is characterized by a Differential Scanning Calorimetry (DSC) trace substantially in accordance with FIG. 2.

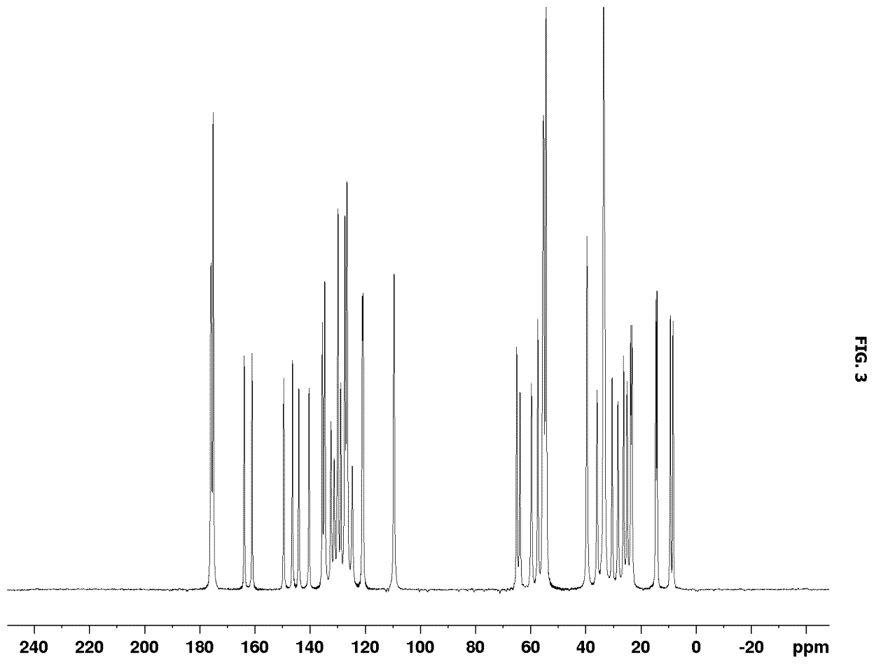

In one embodiment, Form I is characterized by a ¹³C solid-state NMR (SSNMR) spectrum substantially in accordance with FIG. 3.

In one embodiment, Form I is characterized by a ¹³C SSNMR spectrum comprising at least three or at least four carbon peaks selected from the group consisting of about 161.0, 140.4, 124.7, 65.0, 63.8, 35.8, and 30.4 ppm. In one embodiment, Form I is characterized by a ¹³C SSNMR spectrum comprising carbon peaks at about 161.0, 140.4, 124.7, 65.0, 63.8, 35.8, and 30.4 ppm.

In another embodiment, Form I is characterized by single crystal X-ray diffraction (XRD) resulting in the following unit cell parameters:
a=9.0428(5) Å; b=18.5813(9) Å; c=35.320(2) Å; α=β=γ=90°;
V 5934.7(6) Å3;
Space group P2₁2₁2₁;
Drug molecules/unit cell 8;
Z′=2 wherein Z′ is the number of drug molecules per asymmetric unit;
Density (calculated) 1.224 g/cm³.

The present disclosure also provides a method for preparing Form I of linerixibat comprising crystallizing linerixibat in a solvent mixture of water and an organic solvent. In one embodiment, the organic solvent is acetonitrile (MeCN). In one embodiment, the organic solvent is 1-butanol.

In one embodiment, the present disclosure provides a method of preparing Form I of linerixibat comprising crystallizing linerixibat in solvent mixture of MeCN and water. In some embodiments, the method of preparing Form I is carried out on a commercial scale (e.g., greater than 1 kg, 5 kg, or 10 kg).

### Form III of Linerixibat

The present disclosure also provides Form III of linerixibat. Form III of linerixibat is a non-solvated crystalline form that melts with decomposition at onset temperature about 203° C. and peak temperature about 206° C. During the polymorph screen study, this form was primarily obtained from desolvation of several alcoholic solvates, with solvents such as 2-propanol, ethanol, trifluoroethanol or methanol.

In one embodiment, Form III is characterized by an XRPD pattern comprising at least three, at least four, at least five, at least six, or at least seven diffraction angles, when measured using Cu K_{α} radiation, selected from the group consisting of about 5.2, 7.1, 10.4, 13.3, 15.7, 19.1, 20.9, and 21.3 degrees 2θ. In one embodiment, Form III is characterized by an XRPD pattern comprising at least three, at least four, at least five, at least six, or at least seven diffraction angles (d-spacing), when measured using Cu K_{α} radiation, selected from the group consisting of about 5.2 (17.0), 7.1 (12.5), 10.4 (8.5), 13.3 (6.6), 15.7 (5.7), 19.1 (4.6), 20.9 (4.2), and 21.3 (4.2) degrees 2θ.

In one embodiment, Form III is is characterized by an XRPD pattern comprising at least three or at least four diffraction angles, when measured using Cu K_{α} radiation, selected from the group consisting of about 5.2, 7.1, 10.4, 13.3, 15.7, 19.1, 20.9, and 21.3 degrees 2θ.

In one embodiment, Form III is characterized by an XRPD pattern comprising at least four diffraction angles, when measured using Cu K_{α} radiation, selected from the group consisting of about 5.2, 7.1, 10.4, 19.1, and 20.9 degrees 2θ. In one embodiment, Form III is characterized by an XRPD pattern comprising at least four diffraction angles (d-spacing), when measured using Cu K_{α} radiation, selected from the group consisting of about 5.2 (17.0), 7.1 (12.5), 10.4 (8.5), 19.1 (4.6), and 20.9 (4.2) degrees 2θ.

In one embodiment, Form III is characterized by an XRPD pattern comprising at least three diffraction angles, when measured using Cu K_{α} radiation, selected from the group consisting of about 5.2, 7.1, 10.4, 19.1, and 20.9 degrees 2θ. In one embodiment, Form III is characterized by an XRPD pattern comprising at least three diffraction angles (d-spacing), when measured using Cu K_{α} radiation, selected from the group consisting of about 5.2 (17.0), 7.1 (12.5), 10.4 (8.5), 19.1 (4.6), and 20.9 (4.2) degrees 2θ.

In one embodiment, Form III is characterized by an XRPD pattern comprising at least three diffraction angles, when measured using Cu K_{α} radiation, at about 5.2, 7.1, 10.4, and 20.9 degrees 2θ. In one embodiment, Form III is characterized by an XRPD pattern comprising at least three diffraction angles, when measured using Cu K_{α} radiation, selected from the group consisting of about 5.2, 7.1, 10.4, and 20.9 degrees 2θ. In one embodiment, Form III is characterized by an XRPD pattern comprising at least three diffraction angles (d-spacing), when measured using Cu K_{α} radiation, selected from the group consisting of about 5.2 (17.0), 7.1 (12.5), 10.4 (8.5), and 20.9 (4.2) degrees 2θ.

In one embodiment, Form III is characterized by an XRPD pattern comprising three diffraction angles, when measured using Cu K_{α} radiation, at about 5.2, 10.4, and 20.9 degrees 2θ. In one embodiment, Form III is characterized by an XRPD pattern comprising three diffraction angles (d-spacing), when measured using Cu K_{α} radiation, at about 5.2 (17.0), 10.4 (8.5), and 20.9 (4.2) degrees 2θ. In one embodiment, Form III is characterized by an XRPD pattern comprising three diffraction angles, when measured using Cu K_{α} radiation, at about 5.2, 7.1, and 10.4 degrees 2θ. In one embodiment, Form III is characterized by an XRPD pattern comprising three diffraction angles (d-spacing), when measured using Cu K_{α} radiation, at about 5.2 (17.0), 7.1 (12.5), and 10.4 (8.5) degrees 2θ.

In one embodiment, Form II is characterized by an X-ray powder diffraction (XRPD) pattern comprising at least three, at least four, at least five, at least six, at least seven, at least eight, or at least nine diffraction angles, when measured using Cu K_{α} radiation, selected from the diffraction angles shown in Table 2.

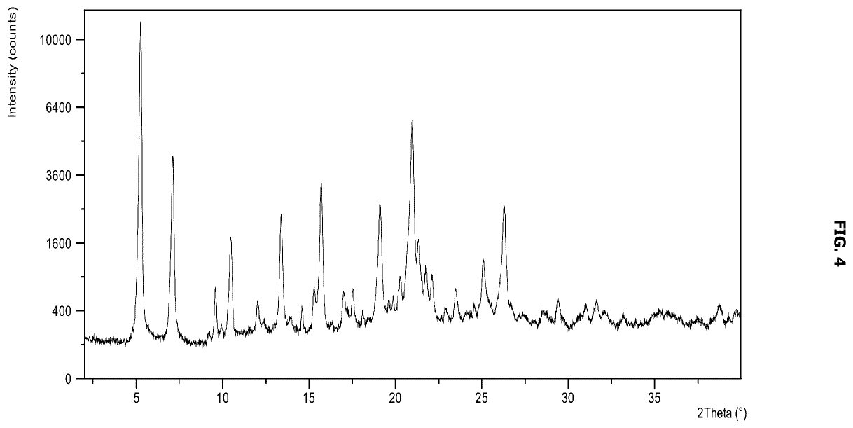

In one embodiment, From III is characterized by an XRPD pattern substantially in accordance with FIG. 4.

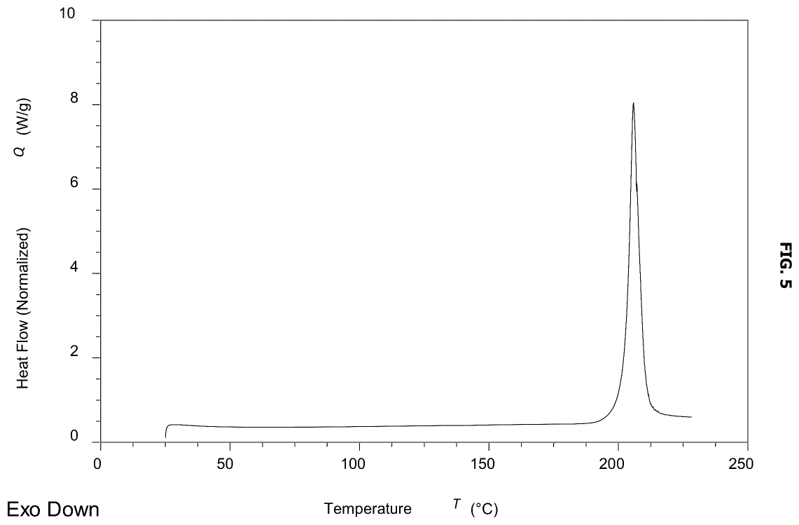

In one embodiment, Form III is characterized by a Differential Scanning Calorimetry (DSC) trace substantially in accordance with FIG. 5.

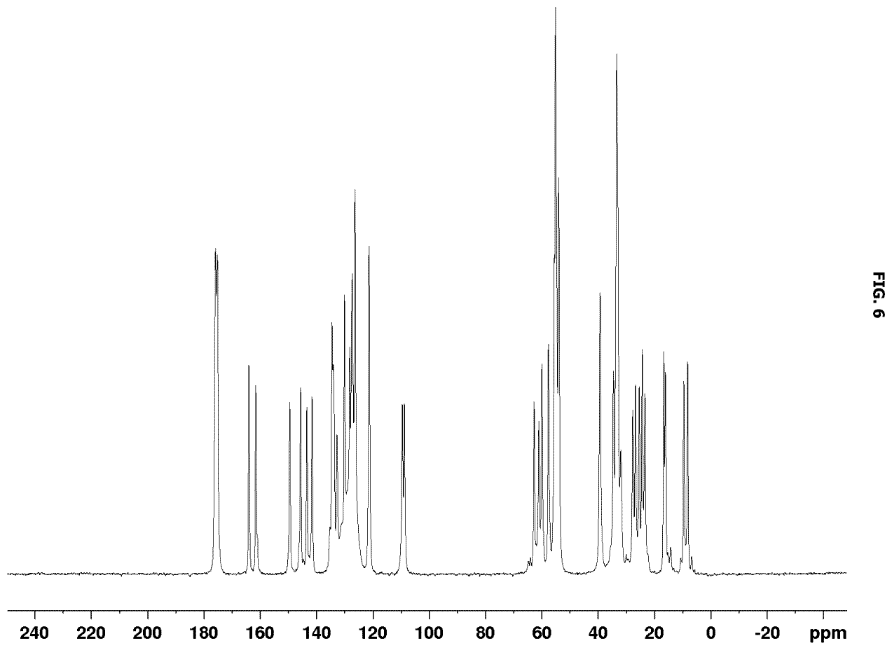

In one embodiment, Form III is characterized by a ¹³C solid-state NMR (SSNMR) spectrum substantially in accordance with FIG. 6.

In one embodiment, Form III is characterized by a ¹³C SSNMR spectrum comprising at least three or at least four carbon peaks selected from the group consisting of about 161.6, 145.6, 141.6, 62.7, 34.5, 24.3, 16.7, and 16.0 ppm. In one embodiment, Form III is characterized by a ¹³C SSNMR spectrum comprising carbon peaks at about 161.6, 145.6, 141.6, 62.7, 34.5, 24.3, 16.7, and 16.0 ppm.

### Form II of Linerixibat

The present disclosure also provides Form II of linerixibat. Form II of linerixibat is a non-solvated crystalline form that melts with decomposition at onset temperature about 205° C. and peak temperature about 206° C. During the polymorph screen study, this form also contained a mixture of other components, generated from evaporation of aqueous organic solvents and from a slurry in dichloromethane.

In one embodiment, Form II is characterized by an XRPD pattern comprising at least three, at least four, at least five, at least six, at least seven or eight diffraction angles (d-spacing), when measured using Cu K_{α} radiation, selected from the group consisting of about 5.1, 6.2, 7.8, 10.1, 11.7, 13.1, 14.4 and 17.3 degrees 2θ, for example selected from the group consisting of about 5.1 (17.5), 6.2 (14.4), 7.8 (11.3), 10.1 (8.7), 11.7 (7.6), 13.1 (6.8), 14.4 (6.1) and 17.3 (5.1) degrees 2θ.

In one embodiment, Form II is characterized by an XRPD pattern comprising at least four or at least three diffraction angles (d-spacing), when measured using Cu K_{α} radiation, selected from the group consisting of about 6.2, 7.8, 10.1, 13.1, 14.4 and 17.3 degrees 2θ, for example selected from the group consisting of about 6.2 (14.4), 7.8 (11.3), 10.1 (8.7), 13.1 (6.8), 14.4 (6.1) and 17.3 (5.1) degrees 2θ.

In one embodiment, Form II is characterized by an XRPD pattern comprising at least four diffraction angles (d-spacing), when measured using Cu K_{α} radiation, selected from the group consisting of about 6.2, 7.8, 11.7, 13.1 and 14.4 degrees 2θ, for example selected from the group consisting of about 6.2 (14.4), 7.8 (11.3), 11.7 (7.6) 13.1 (6.8) and 14.4 (6.1) degrees 2θ.

In one embodiment, Form II is characterized by an XRPD pattern comprising diffraction angles (d-spacing), when measured using Cu K_{α} radiation, at about 6.2, 7.8 and 10.1 degrees 2θ, for example at about 6.2 (14.4), 7.8 (11.3) and 10.1 (8.7) degrees 2θ.

In one embodiment, Form II is characterized by an X-ray powder diffraction (XRPD) pattern comprising at least three, at least four, at least five, at least six, at least seven, at least eight, or at least nine diffraction angles, when measured using Cu K_{α} radiation, selected from the diffraction angles shown in Table 3.

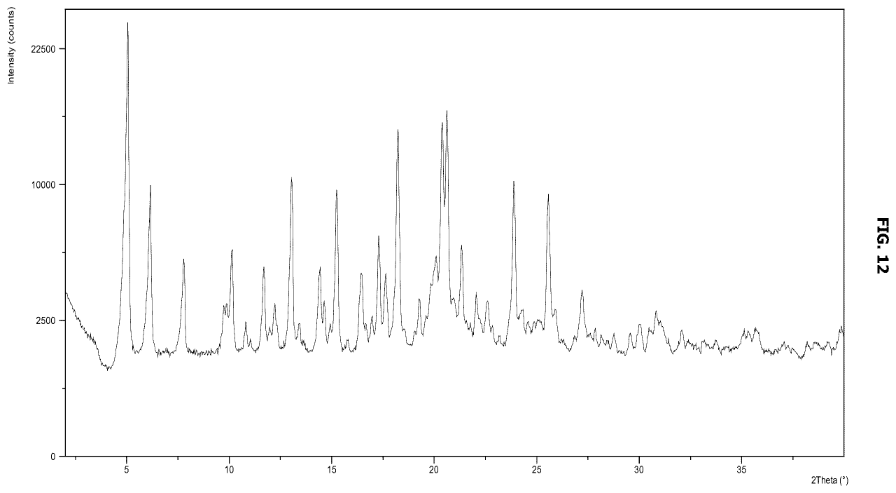

In one embodiment, From II is characterized by an XRPD pattern substantially in accordance with FIG. 12.

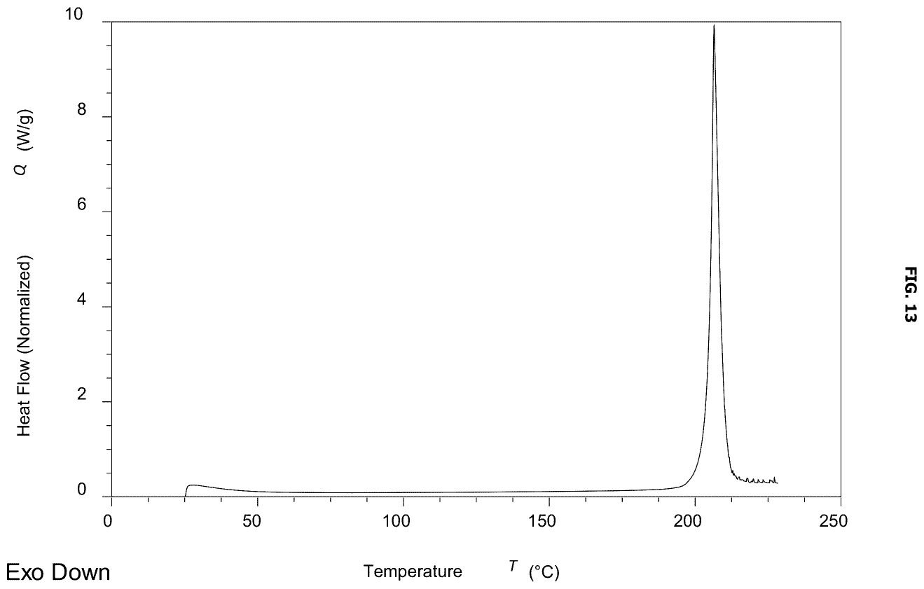

In one embodiment, Form II is characterized by a Differential Scanning Calorimetry (DSC) trace substantially in accordance with FIG. 13.

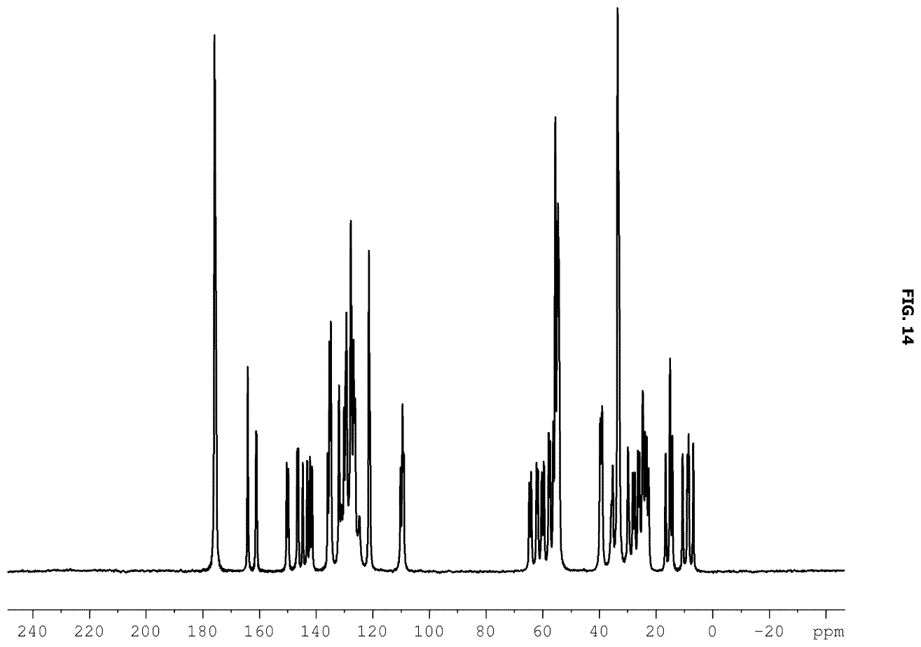

In one embodiment, Form II is characterized by a ¹³C solid-state NMR (SSNMR) spectrum substantially in accordance with FIG. 14.

### Form IV of Linerixibat

The present disclosure also provides Form IV of linerixibat. Form IV of linerixibat is a non-solvated crystalline form that melts with decomposition at onset temperature about 195° C. and peak temperature about 200° C.

In one embodiment, Form IV is characterized by an XRPD pattern comprising at least three, at least four, at least five, at least six, or at least seven diffraction angles (d-spacing), when measured using Cu K_{α} radiation, selected from the group consisting of about 5.1, 10.1, 12.2, 15.1, 20.2, 25.3 and 30.5 degrees 2θ, for example selected from the group consisting of about 5.1 (17.5), 10.1 (8.8), 12.2 (7.3), 15.1 (5.9), 20.2 (4.4), 25.3 (3.5) and 30.5 (2.9) degrees 2θ.

In one embodiment, Form IV is characterized by an X-ray powder diffraction (XRPD) pattern comprising at least three, at least four, at least five, at least six, at least seven, at least eight, or at least nine diffraction angles, when measured using Cu K_{α} radiation, selected from the diffraction angles shown in Table 4.

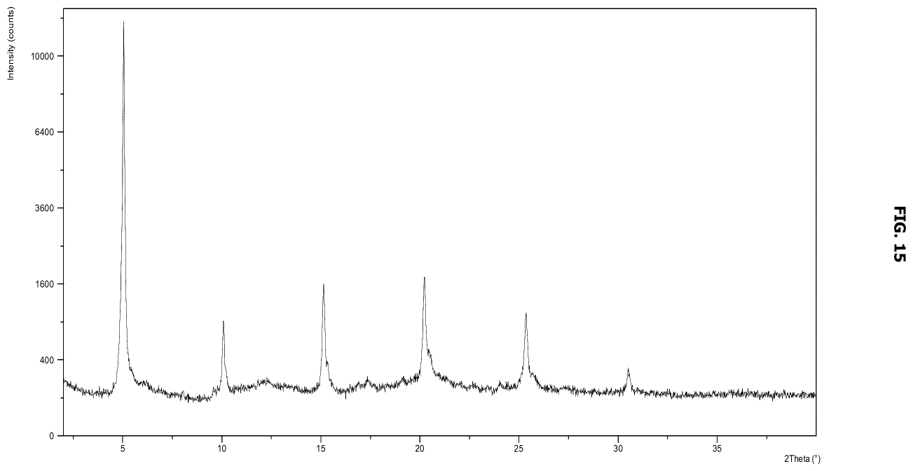

In one embodiment, From IV is characterized by an XRPD pattern substantially in accordance with FIG. 15.

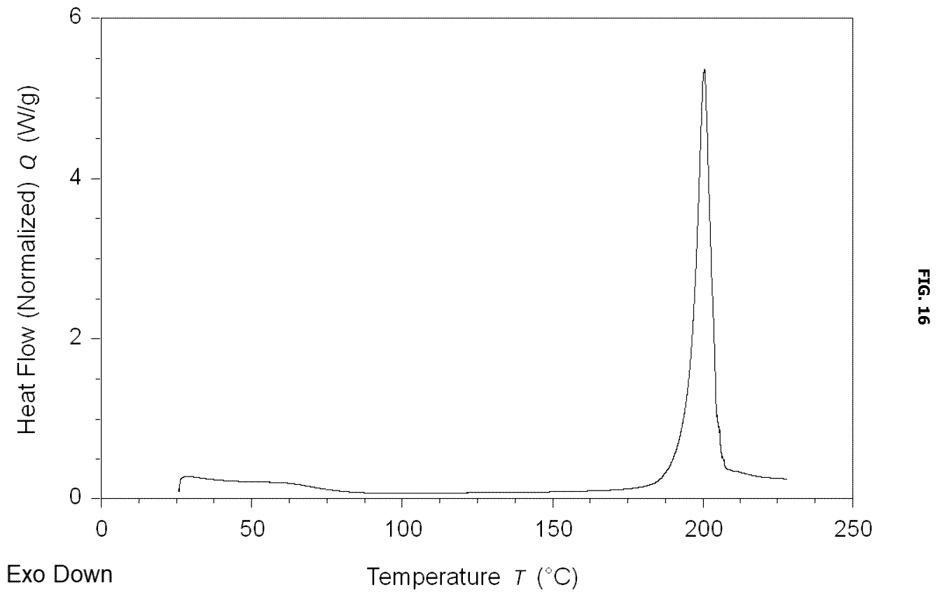

In one embodiment, Form IV is characterized by a Differential Scanning Calorimetry (DSC) trace substantially in accordance with FIG. 16.

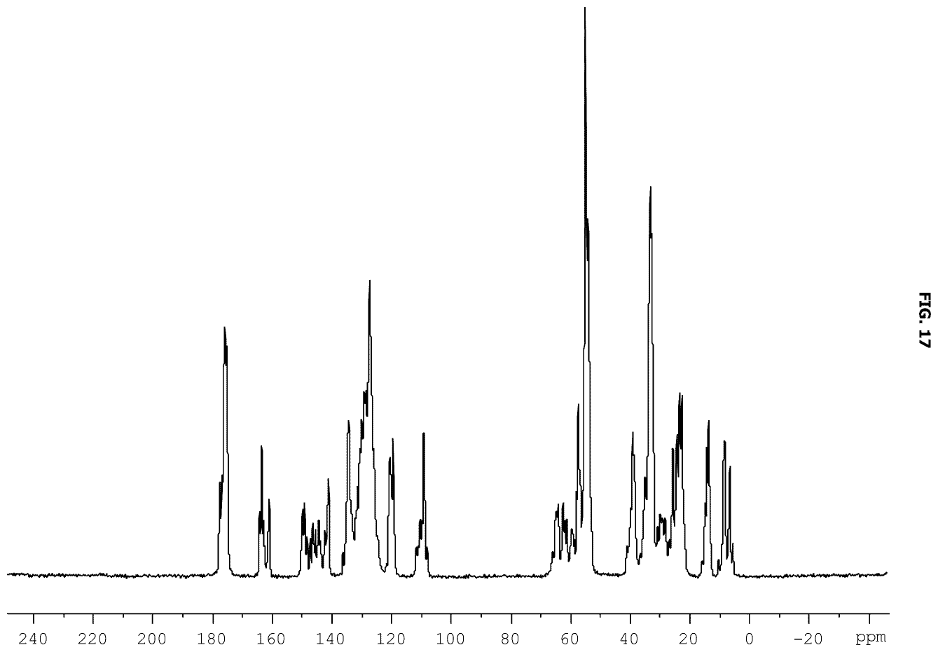

In one embodiment, Form IV is characterized by a ¹³C solid-state NMR (SSNMR) spectrum substantially in accordance with FIG. 17.

### Form V of Linerixibat

The present disclosure also provides Form V of linerixibat. Form V of linerixibat is a non-solvated crystalline form that melts with decomposition at onset temperature about 198° C. and peak temperature about 201° C.

In one embodiment, Form V is characterized by an XRPD pattern comprising at least three, at least four, at least five, at least six, or at least seven diffraction angles (d-spacing), when measured using Cu K_{α} radiation, selected from the group consisting of about 5.3, 7.1, 9.5, 10.7, 12.2, 15.2, 15.8, 17.2, 17.5, 19.0, 19.5, 19.7, 20.3, 20.5, 21.1, 21.6, 23.9, 24.4, 24.8, 25.6 and 26.5, for example selected from the group consisting of about 5.3 (16.8), 7.1 (12.5), 9.5 (9.3), 10.7 (8.2), 12.2 (7.3), 15.2 (5.8), 15.8 (5.6), 17.2 (5.2), 17.5 (5.1), 19.0 (4.7), 19.5 (4.5), 19.7 (4.5), 20.3 (4.4), 20.5 (4.3), 21.1 (4.2), 21.6 (4.1), 23.9 (3.7), 24.4 (3.6), 24.8 (3.6), 25.6 (3.5) and 26.5 (3.4) degrees 2θ.

In one embodiment, Form V is characterized by an XRPD pattern comprising at least four diffraction angles (d-spacing), when measured using Cu Kₐ radiation, selected from the group consisting of about 5.3, 10.7, 15.8 and 17.2, for example selected from the group consisting of about 5.3 (16.8), 10.7 (8.2), 15.8 (5.6) and 17.2 (5.2) degrees 2θ. In one embodiment, Form V is characterized by an XRPD pattern comprising three diffraction angles (d-spacing), when measured using Cu K_{α} radiation, at about 5.3, 10.7 and 15.8, for example at about 5.3 (16.8), 10.7 (8.2) and 15.8 (5.6) degrees 2θ.

In one embodiment, Form V is characterized by an X-ray powder diffraction (XRPD) pattern comprising at least three, at least four, at least five, at least six, at least seven, at least eight, or at least nine diffraction angles, when measured using Cu K_{α} radiation, selected from the diffraction angles shown in Table 5.

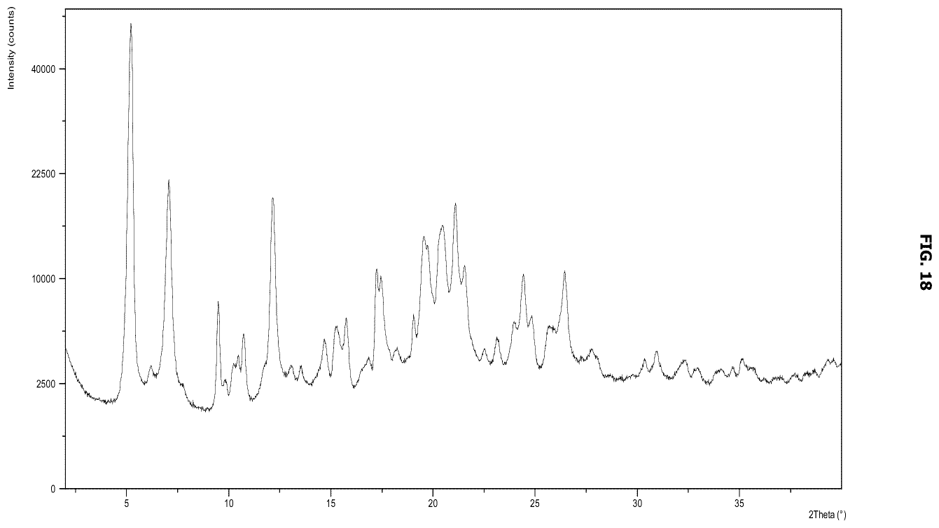

In one embodiment, From V is characterized by an XRPD pattern substantially in accordance with FIG. 18.

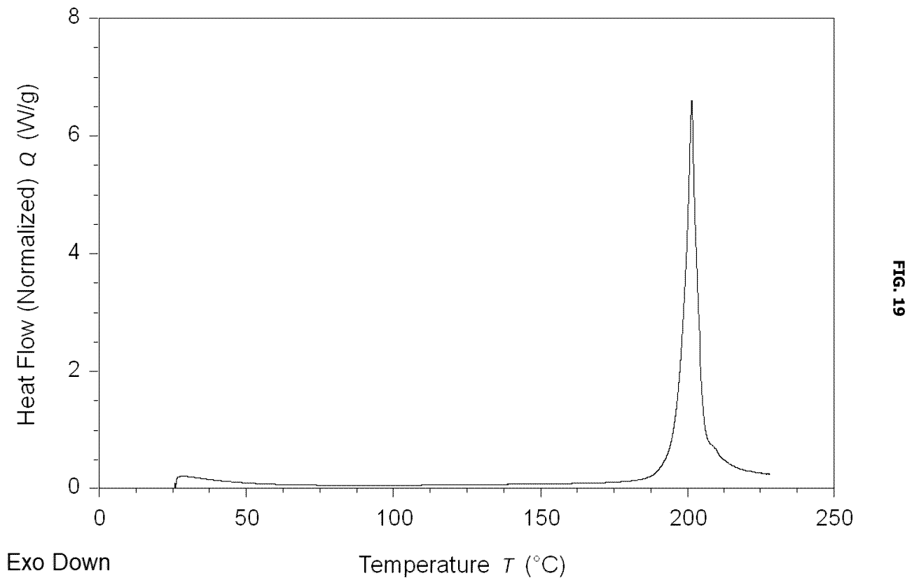

In one embodiment, Form V is characterized by a Differential Scanning Calorimetry (DSC) trace substantially in accordance with FIG. 19.

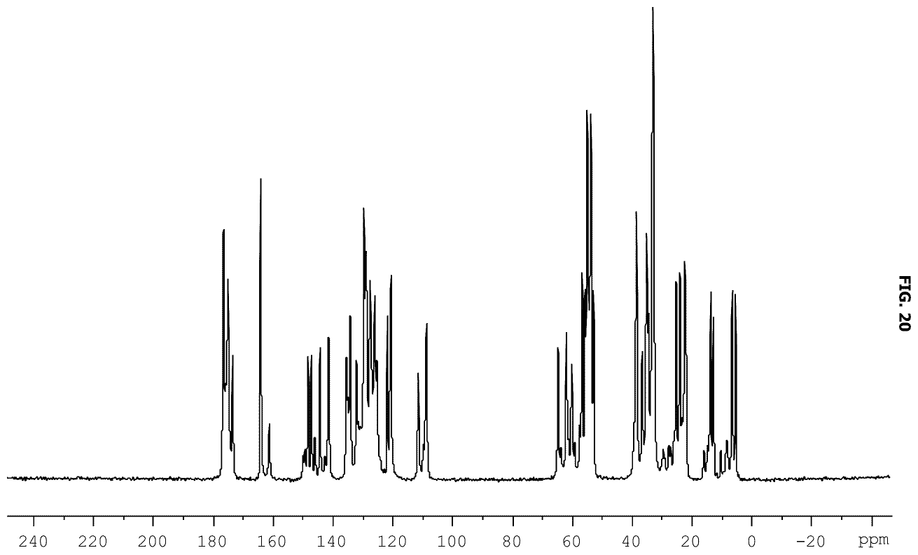

In one embodiment, Form V is characterized by a ¹³C solid-state NMR (SSNMR) spectrum substantially in accordance with FIG. 20.

### Thermal Data

Table A provides melt onset and melt peak data measured by DSC, for Forms I, II, III, IV and V. The DSC thermograms were obtained using a TA discovery Q2500.

TABLE A
Thermal data summary

| GSK2330672B | Reported Melt | Reported Melt Peak |
|---|---|---|
| Form | Onset (° C.) | Temperatures (° C.) |
| I | 205.6 (206) | 208.6 (209) |
| II | 204.7 (205) | 206.5 (206) |
| III | 202.6 (203) | 205.8 (206) |
| IV | 195.2 (195) | 200.4 (200) |
| V | 197.5 (198) | 201.3 (201) |

The present invention also provides a crystalline form of an IBAT inhibitor (linerixibat) which has a melt onset of between about 202° C.-206° C. The DSC thermograms are obtained using a TA discovery Q2500.

### Composition Comprising Form I and Form III

The present disclosure further provides a composition comprising Form III. In some embodiments, the composition comprises Form I and Form III.

As used herein, the term “drug substance” is as currently defined by the FDA (https://www.ecfr.qov/cai-bin/text-idx?SID=6bb682592d11076ada7004b2b3cd73ae&mc=true&node=se21.5.314 13&rgn=div8), i.e. an active ingredient that is intended to furnish pharmacological activity or other direct effect in the diagnosis, cure, mitigation, treatment, or prevention of disease or to affect the structure or any function of the human body, but does not include intermediates used in the synthesis of such ingredient.

As used herein, the term “drug product” is as currently defined by the FDA (https://www.ecfr.qov/cgi-bin/text-idx?SID=6bb682592d11076ada7004b2b3cd73ae&mc=true&node=se21.5.314 13&rgn=div8), i.e. a finished dosage form, e.g., tablet, capsule, or solution, that contains a drug substance, generally, but not necessarily, in association with one or more other ingredients.

In one embodiment, the invention provides a composition in the form of a drug substance. In another embodiment, the invention provides a composition in the form of a drug product.

Conversion of Form I to Form III was observed when Form I was subjected to mechanical stress and/or compaction (e.g., during the tablet compression process or as a result of mechanical stress during the manufacture of Form I), thus forming a mixture of Form I and Form III. Experimental determination showed that Form III was present in compacts using input drug substance of Form I manufactured from different synthetic routes. A likely mechanism for this form change, based on the crystal structure and particle morphology analysis of Form I, combined with XRPD indexing of Form III, also supported the fact that Form III can be obtained by compression of Form I.

As used herein, when the term “an amount” or “by weight” is used to describe an amount or weight of a polymorphic Form of linerixibat (for example an amount of Form 3), the amount or weight referred to is that of the unformulated “drug substance” (as defined herein) rather than that of the finished “drug product” (also as defined herein).

In one embodiment, Form III and Form I are present together, wherein Form III is present in an amount of about 1% to 100%, about 5% to 60%, about 10% to 50% or about 10% to 40% by weight, for example in an amount of less than about 10%, less than about 20%, less than about 30%, less than about 40%, less than about 50%, or less than about 60% by weight. In one embodiment, Form III is present in an amount of about 1% to 100%, about 5% to 60%, or about 10% to 50% by weight. In one embodiment, there is provided a composition comprising a mixture of crystalline Form I and III of linerixibat wherein Form III is present in an amount of about 1% to 100%, about 5% to 60%, about 10% to 50% by weight or about 10% to 40% of the linerixibat drug substance component of the composition. In another embodiment, there is provided a composition comprising a mixture of crystalline Form I and III of linerixibat wherein Form III is present in an amount of less than or equal to about 40% by weight of the linerixibat drug substance component of the composition. In another embodiment, linerixibat is present the composition in an amount of about 40 mg.

In another embodiment, Form III and Form I are present together, wherein Form I is present in an amount of about 1% to 99%, about 40% to 95%, about 50% to 90% or about 60% to 90% by weight. In one embodiment, Form I is present in an amount of about 1% to 99%, about 40% to 95%, or about 50% to 90% by weight. In one embodiment, Form III and Form I are present together, wherein Form I is present in an amount of about 1%, 2%, 3%, 4%, 5%, 10%, 20%, 30%, 40%, 50%, 60%, 65%, 70%, 75%, 80%, 85%, 90%, 95%, 96%, 97%, 98%, or 99% by weight, or in a range between any two of the preceding percentages. In one embodiment, Form III and Form I are present together, wherein Form I is present in an amount of about 90% to 99% by weight. In one embodiment, Form III and Form I are present together, wherein Form I is present in an amount of about 60% to 99% by weight. In one embodiment, Form III and Form I are present together, wherein Form I is present in an amount of about 50% to 99% by weight. In one embodiment, Form III and Form I are present together, wherein Form I is present in an amount of about 60% to 90% by weight.

In one embodiment, there is provided a composition comprising a mixture of crystalline Form I and III of linerixibat wherein Form I is present in an amount of about 1% to 99%, about 40% to 95%, about 50% to 90% or about 60% to 90% by weight of the linerixibat drug substance component of the composition. In another embodiment, linerixibat is present the composition in an amount of about 40 mg.

In one embodiment, Form III and Form I are present together, wherein Form I is present in an amount of at least about 40%, at least about 50%, at least about 60%, at least about 70%, at least about 80% or at least about 90% by weight.

In one embodiment, the present invention provides a pharmaceutical composition, for example, an oral dosage form (e.g., a tablet or a capsule) comprising Form I and Form III of linerixibat, wherein Form III is present in an amount of about 1% to 100% by weight of the linerixibat drug substance component of the composition. In one embodiment, the pharmaceutical composition is a tablet and Form III is present in an amount of less than or equal to about 40%, less than or equal to about 50%, or less than or equal to about 60% by weight, of the linerixibat drug substance component of the composition. In one embodiment, the pharmaceutical composition is a tablet and the linerixibat drug substance component of the composition comprises substantially pure Form III. In one embodiment, Form III is present in the linerixibat drug substance component of the tablet in an amount of less than about 50% or less than about 40% by weight.

In some embodiments, as a person having ordinary skill in the art will understand, a particular linerixibat polymorph is characterized by any combination of two or more sets of the analytical data characterizing the aforementioned embodiments. For example, in one embodiment, Form I is characterized by an X-ray powder diffraction (XRPD) pattern substantially in accordance with FIG. 1 and a ¹³C solid-state NMR (SSNMR) spectrum substantially in accordance with FIG. 3. In another embodiment, Form III is characterized by an X-ray powder diffraction (XRPD) pattern substantially in accordance with FIG. 4 and a ¹³C solid-state NMR (SSNMR) spectrum substantially in accordance with FIG. 6.

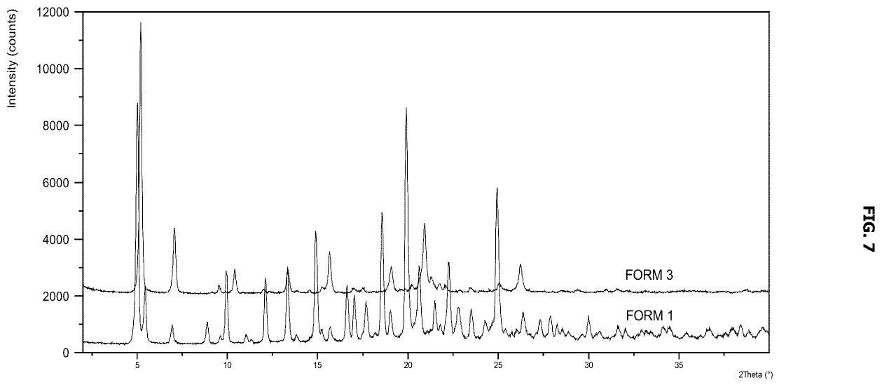

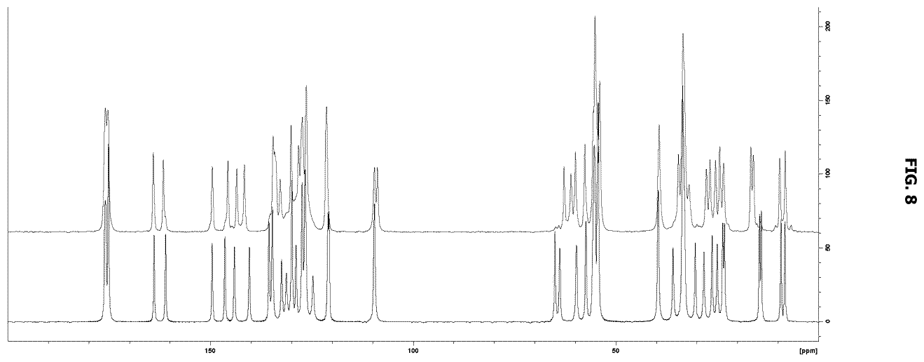

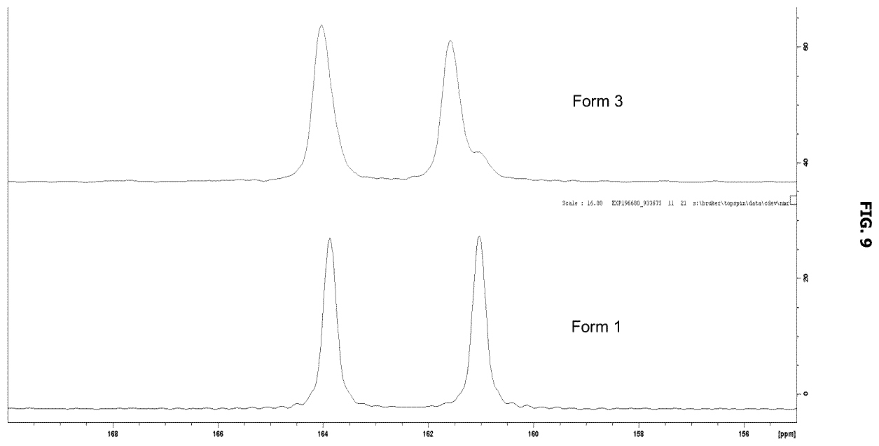

Forms I and III are readily distinguishable by XRPD. The overlay of their full diffractograms are shown in FIG. 7. There are additional peaks at approximately 5.2, 7.1, and 10.4° 2θ that are not present in the Form I diffractogram but are present in the Form III diffractogram. Forms I and III are also readily distinguishable by ¹³C SSNMR spectroscopy. Comparison of the ¹³C SSNMR spectra of Forms I and III is shown in FIG. 8. Doubling of some resonances in the spectra of both Form I and Form III indicates Z′=2 (number of molecules per asymmetric unit), which is consistent with the single crystal structure determined by X-ray diffraction. An expanded aromatic spectral region shows, as an example, characteristic peaks of Forms I and III at about 161.0 ppm and about 161.6 ppm, respectively (FIG. 9). The characteristic resonances in the ¹³C SSNMR spectra, e.g., at chemical shifts ca. 161 ppm, can be used for assessing the levels of Form III in the composition of the drug substance and drug product.

Key properties of Form I and Form III were analyzed and listed in Table B. As has been reported to the U.S. Food and Drug Administration (FDA), it was surprising to find that the properties of the two solid state forms are similar, with comparable melting points, similar solubilities at biorelevant pH values, and no notable differences with respect to stability.

TABLE B

| Property | Conclusion Summary |
|---|---|
| Solubility | Forms I and III have very similar solubilities at all investigated physiologically |
| | relevant pH values (1.6 to 6.5). |
| Stability of Drug | A mix of Form I and Form III demonstrated no change of form or drug-related |
| Substance | impurity content after 1 month of storage at 40° C./75% RH and 50° C./ambient |
| | RH by HPLC (high performance liquid chromatography), XRPD and SSNMR. |
| | Laboratory isolated Form III drug substance demonstrated no change in form |
| | or drug-related impurity content after 1 month storage at 50° C./ambient |
| | RH when assessed by HPLC, XRPD and SSNMR. |
| Stability of Drug | There was no apparent change in Form III content of tablets formulated with |
| Product | Form I drug substance when stored for 52 months and 36 months, respectively, |
| | at 5° C./ambient RH and 30° C./65% RH by XRPD and |
| | SSNMR. No significant change in dissolution was observed either. |
| Melting Point | Forms I and III demonstrate very similar melt onsets (about 205.6° C. and |
| by DSC | about 202.6° C. respectively) |

In addition, linerixibat exhibits minimal systemic absorption, and both Form I and Form III display similar solubility and dissolution behaviour, including in biorelevant media, and will be fully in solution at the site of action. Therefore, it is not expected that the ratio of Forms I and III in the drug substance at the intended clinical dose, at the point of administration, will impact the in vivo performance in terms of product safety, performance or efficacy. It is not deemed necessary to take measures to avoid or control the form change from Form I to Form III during manufacture of the pharmaceutical composition.

Hence, in one aspect, the present invention provides a novel pharmaceutical composition comprising Form I and Form III of linerixibat.

The present invention also provides a crystalline form of linerixibat which demonstrates no change in form or drug-related impurity content after 1 month of storage at 40° C./75% RH and 50° C./ambient RH by HPLC, XRPD and SSNMR.

### XRPD (X-Ray Powder Diffraction)

An XRPD pattern will be understood to comprise a diffraction angle (expressed in “degrees 2θ” or “° 2θ”) of “about” a value specified herein when the XRPD pattern comprises a diffraction angle within +0.2 degrees 2θ of the specified value, i.e. the margin of error. Thus, in certain embodiments the margin of error in respect of XRPD diffraction angles will be within ±0.2 degrees 2θ of the specified value. In other embodiments, the margin of error in respect of XRPD diffraction angles will be within ±0.1 degrees 2θ of the specified value. Further, it is well known and understood to those skilled in the art that the apparatus employed, humidity, temperature, orientation of the powder crystals, and other parameters such as displacement of the sample and other experimental error such as sample preparation height involved in obtaining an X-ray powder diffraction (XRPD) pattern may cause some variability in the appearance, intensities, and positions of the lines in the diffraction pattern.

The term “XRPD” is used herein interchangeably with the term “PXRD.”

The X-ray powder diffraction patterns provided herein were produced using silicon wafer reflection XRPD.

An X-ray powder diffraction pattern that is “substantially in accordance” with that of FIG. 1, FIG. 4, FIG. 12, FIG. 15 or FIG. 18 provided herein is an XRPD pattern that would be considered by one skilled in the art to represent a compound possessing the same crystal form as the compound that provided the XRPD pattern of FIG. 1, FIG. 4, FIG. 12, FIG. 15 or FIG. 18. That is, the XRPD pattern may be identical to that of FIG. 1, FIG. 4, FIG. 12, FIG. 15 or FIG. 18, or more likely it may be somewhat different. Such an XRPD pattern may not necessarily show each of the lines of any one of the diffraction patterns presented herein, and/or may show a slight change in appearance, intensity, or a shift in position of said lines resulting from differences in the conditions involved in obtaining the data. A person skilled in the art is capable of determining if a sample of a crystalline compound has the same form as, or a different form from, a form disclosed herein by comparison of their XRPD patterns. For example, one skilled in the art can overlay an XRPD pattern of a sample containing linerixibat, with FIG. 1 and, using expertise and knowledge in the art, readily determine whether the XRPD pattern of the sample is substantially in accordance with the XRPD pattern of Form I of linerixibat. If the XRPD pattern is substantially in accordance with FIG. 1, the sample form can be readily and accurately identified as having the same form as Form I.

¹³C SSNMR (¹³C Solid State Nuclear Magnetic Resonance)

Similarly, an SSNMR spectrum will be understood to comprise a peak of “about” a value specified herein, when the ¹³C SSNMR spectrum comprises an isotopic chemical shift value within ±0.1 ppm of the specified peak value, i.e. the margin of error. Thus, in certain embodiments the margin of error in respect of ¹³C SSNMR spectrum peaks will be within ±0.1 ppm of the specified peak value.

It is well known and understood to those skilled in the art that the apparatus employed, humidity, temperature, and other parameters such as field strength and spinning frequencies involved in obtaining a SSNMR spectrum may cause some variability in the appearance, intensities, and positions of the peaks in the spectrum. A ¹³C SSNMR spectrum that is “substantially in accordance” with that of FIG. 3, FIG. 6, FIG. 14, FIG. 17 or FIG. 20 provided herein is a ¹³C SSNMR spectrum that would be considered by one skilled in the art to represent a compound possessing the same crystal form as the compound that provided the ¹³C SSNMR spectrum of FIG. 3, FIG. 6, FIG. 14, FIG. 17 or FIG. 20. That is, the ¹³C SSNMR spectrum may be identical to that of FIG. 3, FIG. 6, FIG. 14, FIG. 17 or FIG. 20, or more likely it may be somewhat different. A person skilled in the art is capable of determining if a sample of a crystalline compound has the same form as, or a different form from, a form disclosed herein by comparison of their ¹³C SSNMR spectra, for example by overlaying them.

### Compound of the Invention

“Compound of the invention” means 3-({[(3R,5R)-3-butyl-3-ethyl-7-(methyloxy)-1,1-dioxido-5-phenyl-2,3,4,5-tetrahydro-1,4-benzothiazepin-8-yl]methyl}amino)pentanedioic acid (i.e., linerixibat), its crystalline forms (including Form I, Form II, Form III, Form IV, Form V), amorphous linerixibat or a mixture of two or more thereof, including a mixture of Form I and Form III.

### Solubility and Dissolution Profile of Linerixibat

Linerixibat is locally acting in the lower intestine, with minimal systemic absorption. The transit time to the site of action in the distal ileum will typically be 3-4 hours before reaching the site of action (range 1-9 hours). The pH of the distal ileum (site of action) is around pH 6.8, and a conservative estimate of free liquid intestinal volume is 100 mL. Therefore, linerixibat will be in solution at the site of action provided that:
Solubility of linerixibat drug substance at intestinal pH is ≥0.4 mg/mL (40 mg dose, 100 mL intestinal volume);
Dissolution of linerixibat drug product is complete in ≤1 hour under simulated intestinal conditions (pH 6.8).

Linerixibat has been shown to be very rapidly releasing at both gastric and intestinal pH (solubility at pH 1.2 and pH 6.8 is >1 mg/mL and shows full release in 5 minutes). A pH of 6.8 represents the most biologically relevant pH for measuring dissolution and is appropriate based upon the locally acting nature of the drug and minimal systemic absorption. Demonstration of very rapid release of linerixibat under these conditions provides assurance of drug product quality and performance.

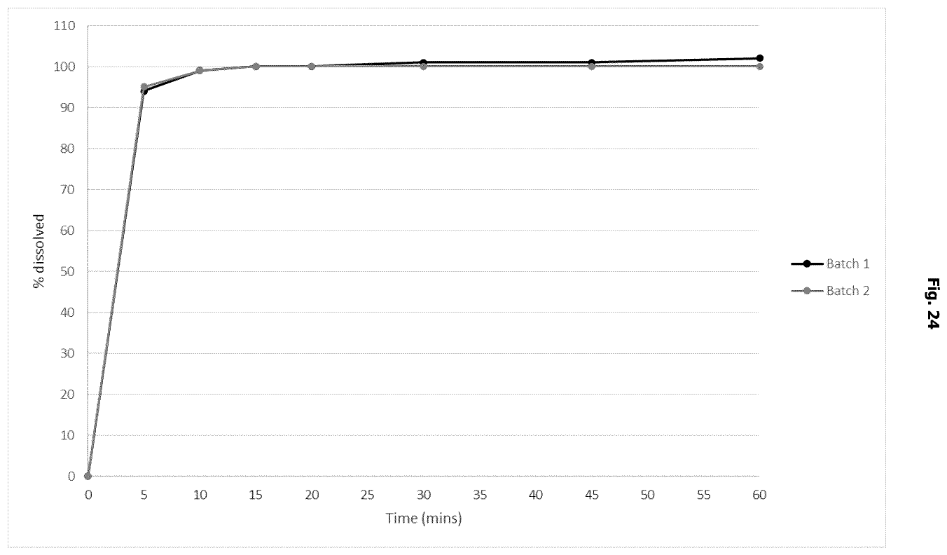

Accordingly, the present invention provides an oral dosage form of linerixibat, characterised in that linerixibat is in a form which has a solubility of ≥0.4 mg/mL at an intestinal pH of about 6.8 and wherein dissolution of the oral dosage form is complete in ≤1 hour. In one embodiment, linerixibat is present in a form which has a solubility of >1 mg/mL at an intestinal pH of about 6.8. In another embodiment, linerixibat is present in a form which has a solubility of >5 mg/mL at an intestinal pH of about 6.8. In a further embodiment, linerixibat is present in a form which has a solubility of >1 mg/mL at a gastric pH of about 1.2. In another embodiment, linerixibat is present in a form which has a solubility of >7 mg/mL at a gastric pH of about 1.2. In one embodiment, linerixibat is present in a form disclosed herein (Form I, Form II, Form III, Form IV, Form V or amorphous linerixibat) or a or a mixture of two or more thereof. In another embodiment, linerixibat is present as crystalline Form I, crystalline Form III, or a mixture thereof. In a further embodiment, linerixibat is present in an amount of about 40 mg. In one embodiment, the oral dosage form of linerixibat is a tablet. In a further embodiment, the oral dosage form is at least 90% in solution in aqueous buffer at about pH 6.8, after 5 mins. In one embodiment, the oral dosage form of linerixibat exhibits a dissolution profile substantially in accordance with FIG. 24. In another embodiment, an oral dosage form of linerixibat is 80-125% bioequivalent to a given oral dosage form disclosed herein.

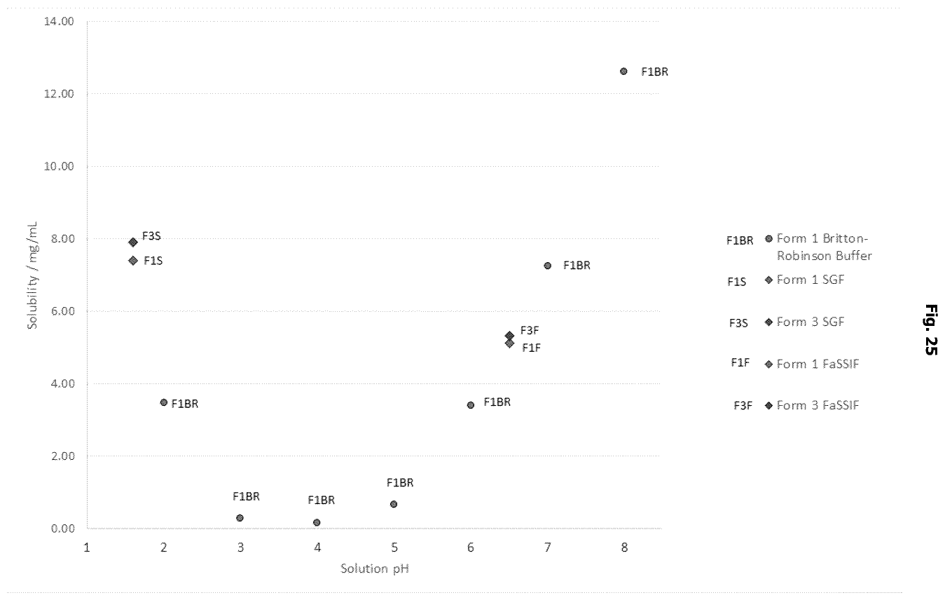

The present invention also provides an IBAT inhibitor which exhibits a solubility profile substantially in accordance with FIG. 25. The present invention also provides an IBAT inhibitor which exhibits a solubility profile 80-125% equivalent to that shown in FIG. 25. In one embodiment, the IBAT inhibitor is linerixibat.

### Use, Method of Treatment, and Pharmaceutical Compositions

The invention provides a method for treating cholestatic pruritus in a patient with primary biliary cholangitis comprising administering to the patient an effective amount of a compound of the invention or a composition comprising an effective amount of a compound of the invention and an optional pharmaceutically acceptable carrier. The method of treating cholestatic pruritus by using linerixibat is described in the literature. See e.g., Hegade, V. S., et al., “Effect of ileal bile acid transporter inhibitor GSK2330672 on pruritus in primary biliary cholangitis: a double-blind, randomised, placebo-controlled, crossover, phase 2a study,” Lancet, 389(10074):1114-112 (2007), which is incorporated herein by reference in its entirety.

As used herein, the term “treatment” refers to alleviating the specified condition, eliminating or reducing one or more symptoms of the condition, slowing or eliminating the progression of the condition, and preventing or delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician. Unless otherwise stated, the amount of a drug or pharmaceutical agent refers to the amount of the free base compound, not the amount of the corresponding pharmaceutically acceptable salt.

The present invention is also directed to a pharmaceutical composition comprising a compound of the invention and a pharmaceutically acceptable carrier. The present invention is further directed to a method of preparing a pharmaceutical composition comprising admixing a compound of the invention and a pharmaceutically acceptable carrier.

“Pharmaceutically acceptable carrier” means any one or more compounds and/or compositions that are of sufficient purity and quality for use in the formulation of the compound of the invention that, when appropriately administered to a human, do not produce an adverse reaction, and that are used as a vehicle for a drug substance (i.e. a compound of the present invention). Carriers may include excipients, diluents, granulating and/or dispersing agents, surface active agents and/or emulsifiers, binding agents, preservatives, buffering agents, lubricating agents, and natural oils. Therefore, in one aspect of the invention, there is provided a pharmaceutical composition comprising the composition of linerixibat in a form disclosed herein, and a pharmaceutically acceptable excipient.

The invention further includes the process for making a pharmaceutical composition comprising mixing a compound of the invention and one or more pharmaceutically acceptable carriers; and includes those compositions resulting from such a process, which process includes conventional pharmaceutical techniques. For example, a compound of the invention may be nanomilled prior to formulation. A compound of the invention may also be prepared by grinding, micronizing or other particle size reduction methods known in the art. The pharmaceutical compositions of the invention may be prepared using techniques and methods known to those skilled in the art. Some of the methods commonly used in the art are described in Remington's Pharmaceutical Sciences (Mack Publishing Company), the entire teachings of which are incorporated herein by reference.

In particular, the compound of the present invention, or corresponding pharmaceutical compositions or formulations used in the present invention may be formulated for administration in any convenient way for use in human or veterinary medicine.

In one embodiment, the pharmaceutical composition is for oral administration. The pharmaceutical compositions may be in the form of tablets, capsules, powders, or granules. In one embodiment, the pharmaceutical composition is a tablet or capsule. In another embodiment, the pharmaceutical composition is a tablet. In another embodiment, Form I and Form III are present together and Form III is present in the tablet in an amount of less than about 50% or less than about 40% by weight.

Tablets and capsules for oral administration in the present invention may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

The present invention provides a method of preparing a pharmaceutical composition comprising linerixibat, wherein the method comprises (1) mixing Form I, Form III, or a mixture of Form I and Form III, or a mixture of two or more of Form I, Form II, Form III, Form IV, Form V and amorphous linerixibat with a pharmaceutically acceptable carrier. In one embodiment, the method further comprises compressing the resulting mixture to form a tablet.

The present invention further provides a method of treating cholestatic pruritus in a patient with primary biliary cholangitis comprising administering to the patient an effective amount of the pharmaceutical composition disclosed herein.

The Examples set forth below are illustrative of the present invention and are not intended to limit, in any way, the scope of the present invention.

### EXPERIMENTAL

The following Examples illustrate the present invention. These Examples are not intended to limit the scope of the present invention, but rather to provide guidance to the skilled artisan to prepare and use the crystalline forms, compositions, and methods of the present invention. While particular embodiments of the present invention are described, the skilled artisan will appreciate that various changes and modifications can be made without departing from the spirit and scope of the invention. Unless otherwise noted, reagents are commercially available or are prepared according to procedures in the literature.

### Example 1

### Form I of Linerixibat and Amorphous Linerixibat

### Example 1a—Preparation of Amorphous Material

### Method 1

Linerixibat (3.04 g) was combined with acetic acid (60 mL) and stirred at ambient temperature to yield a solution which was filtered through a 0.2 μm syringe filter. The filtrate (500 μL) was pipetted into 2 mL vials which were capped and frozen in liquid nitrogen. The vial caps were quickly removed and the vials were lyophilized for three days. XRPD analysis of the product showed no crystalline material. Amorphous linerixibat was used as input for a polymorph screen study.

### Method 2

Amorphous linerixibat may alternatively be made using techniques which are well-known to the skilled artisan, including, but not limited to: i) by means of mechanical impact, for example, ball milling or micronisation; ii) heating followed by quench cooling or heating of a solvate resulting in desolvation; iii) by means of certain solvent-based processes, for example, rotary evaporation, lyophilisation, precipitation or spray-drying.

For example, amorphous linerixibat was generated by lyophilisation during a form screen, and also by ball milling during a form screen using a RETSCH MM200 ball mill, large stainless steel chamber, one large 12 mm and two small 10 mm stainless steel balls with ball milling frequency 25 1/s. This was carried out until an amorphous ‘halo’ (i.e. no significant diffraction angles) by XRPD was observed.

### Example 1b—Preparation of Form I

### Method 1

Form I may be prepared according to the procedures in WO2011/137135, Example 26.

### Method 2

Form I of linerixibat was prepared according to the following procedure at a large scale (>500 g). All charges were based on input linerixibat.

An intermediate grade linerixibat was dissolved in acetonitrile/Water (12 vols/8 vols) at reflux (˜76° C.). The solution was seeded at 70° C. with Form I (2% w/w), cooled to 60° C. over 15 mins and aged at 60° C. for 2 hrs. Water (14 vols) was added over 8 hrs and aged for 1 hr. The suspension was cooled to 20° C. over 1 hr and aged for >30 mins. The slurry was filtered, washed with acetonitrile:water (6:11 v/v) (3.5 vols), then twice with water (2 vols) and blown down with nitrogen 8-18 hrs. Drying was carried out under vacuum at 40-50° C. without agitation until the Karl Fisher measurement (KF) was <10% w/w. The batch was agitated at 4 rpm for 2 mins every 3 hrs until the KF≤1% w/w to provide Form I.

Form I can also be prepared by the above procedure without the step of seeding.

### Method 3

Form I of linerixibat was prepared according to the following procedure at a large scale (>50 kg). A reactor (Reactor 1) was charged with 55.84 kg GSK2330672B (1.0 wt) intermediate grade (IG) followed by acetonitrile (12 vol) and purified water (8 vol). The mixture was heated to reflux (74-79° C.), and held until complete dissolution is observed. The solution was then transferred to a reactor (Reactor 2) that had been pre-heated to 74-79° C. via filter (0.22 μm pipe-line filter). Reactor 1 was rinsed with acetonitrile (MeCN) (0.3 vol) and purified water (0.2 vol), and the solution in Reactor 1 was transferred to Reactor 2 via filter (0.22 μm pipe-line filter). The contents of Reactor 2 were held until complete dissolution is observed. The solution in Reactor 2 was cooled to 69-72° C., then seeded with 2 w/w % (based on pure GSK2330672B input). The suspension was cooled to 58-62° C. within 10-20 min. The suspension was held at 58-62° C. for 2 h. Purified water (14 vol) was added over 8 hrs. After the addition was complete, the slurry was held at 58-62° C. for 60 min, then cooled to 18-25° C. over 50-70 min. The slurry was stirred at 18-25° C. for not less than 30 min, then the suspension filtered under vacuum. The reactor was rinsed with MeCN/water (6/11 v:v, 3.5 vol) and the rinse used to wash the cake. The cake was washed twice with water (2 vol). The cake was blown down with nitrogen and dried at 60° C. under vacuum to give 46.35 kg GSK2330672B Form 1 solid.

### Method 4

10.94 g of GSK2330672B was charged and washed into a vessel using 131 mL of acetonitrile (MeCN) and 88 mL of water. The slurry was then heated to reflux. 4 mL of 3:2 v/v MeCN/water was charged followed by 5.5 mL of 3:2 v/v MeCN/water. The contents were cooled to 70° C., then cooled to 60° C. over 15 minutes and held stirring for 2 hours. 153 mL water was added over 8 hours, the contents were cooled to 20° C. over 1 hour, and held stirring for 1 hour. The product was isolated, washed with 38 mL of 6:11 MeCN/water, then washed twice with 22 mL of water. After deliquoring, the product was dried at 45° C. under vacuum, to give 9.35 g (85.5% w/w) Form I GSK2330672B.

### Example 1c—XRPD of Form I

The X-ray powder diffraction (XRPD) pattern of Form I of linerixibat is shown in FIG. 1 and a summary of the diffraction angle and d-spacings is given in Table 1 below. The XRPD data were acquired on a PANalytical X'Pert Pro powder diffractometer, model PW3040/60 using an X'Celerator detector. The acquisition conditions were:
radiation: Cu K_{α},
generator tension: 40 kV,
generator current: 45 mA,
start angle: 2.0° 2θ,
end angle: 40.0° 2θ,
step size: 0.0167° 2θ,
time per step: 31.75 seconds.

The sample was prepared by mounting a few milligrams of sample on a silicon wafer (zero background plate), resulting in a thin layer of powder.

TABLE 1

| Peak No. | Diffraction Angles, 2θ (°) | d-spacings (Å) |
|---|---|---|
| 1 | 5.0 | 17.5 |
| 2 | 5.5 | 16.2 |
| 3 | 7.0 | 12.7 |
| 4 | 8.9 | 10.0 |
| 5 | 9.6 | 9.2 |
| 6 | 9.9 | 8.9 |
| 7 | 11.0 | 8.0 |
| 8 | 11.3 | 7.8 |
| 9 | 12.1 | 7.3 |
| 10 | 13.3 | 6.6 |
| 11 | 13.8 | 6.4 |
| 12 | 14.9 | 6.0 |
| 13 | 15.2 | 5.8 |
| 14 | 15.7 | 5.6 |
| 15 | 16.6 | 5.3 |
| 16 | 17.0 | 5.2 |
| 17 | 17.7 | 5.0 |
| 18 | 18.2 | 4.9 |
| 19 | 18.6 | 4.8 |
| 20 | 19.0 | 4.7 |
| 21 | 19.9 | 4.5 |
| 22 | 20.6 | 4.3 |
| 23 | 21.5 | 4.1 |
| 24 | 21.8 | 4.1 |
| 25 | 22.3 | 4.0 |
| 26 | 22.7 | 3.9 |
| 27 | 23.5 | 3.8 |
| 28 | 24.2 | 3.7 |
| 29 | 24.9 | 3.6 |
| 30 | 25.4 | 3.5 |
| 31 | 25.7 | 3.5 |
| 32 | 26.0 | 3.4 |
| 33 | 26.4 | 3.4 |
| 34 | 27.3 | 3.3 |
| 35 | 27.8 | 3.2 |
| 36 | 28.3 | 3.2 |
| 37 | 28.5 | 3.1 |
| 38 | 28.9 | 3.1 |
| 39 | 29.6 | 3.0 |
| 40 | 30.0 | 3.0 |
| 41 | 30.6 | 2.9 |
| 42 | 31.6 | 2.8 |
| 43 | 32.0 | 2.8 |
| 44 | 32.9 | 2.7 |
| 45 | 33.2 | 2.7 |
| 46 | 34.1 | 2.6 |
| 47 | 34.5 | 2.6 |
| 48 | 35.4 | 2.5 |
| 49 | 36.5 | 2.5 |
| 50 | 36.7 | 2.4 |
| 51 | 37.5 | 2.4 |
| 52 | 38.0 | 2.4 |
| 53 | 38.4 | 2.3 |
| 54 | 38.9 | 2.3 |
| 55 | 39.6 | 2.3 |

### Example 1d—DSC of Form I

The DSC thermograms in the present application were obtained using a TA discovery Q2500. The sample was weighed into an aluminium pan, a pan lid placed on top and lightly crimped without sealing the pan. The experiment was conducted using a heating rate of 10° C. min⁻¹. The DSC thermogram of Form I is shown in FIG. 2 and Form I has a melt onset at approximately 205.6° C. with a peak temperature at about 208.6° C.

### Example 1e—13C Solid State NMR (13CSSNMR) of Form I

¹³C Solid state NMR data were acquired using a Bruker Avance III NMR spectrometer with an operating ¹H frequency of 500.13 MHz. The spectrometer was equipped with a 4 mm double resonance magic-angle spinning probe operating at a rotation frequency of 8 kHz. Spectra were obtained using cross-polarisation, with a linear power ramp used on the ¹H channel to enhance cross-polarisation efficiency. Spinning sidebands were eliminated by a total sideband suppression sequence. ¹H decoupling was obtained using the SPINAL-64 sequence. ¹³C chemical shifts are referenced to tetramethylsilane at 0 ppm (parts per million), using the carbonyl peak in α-glycine at 176.4 ppm as a secondary reference.

The ¹³C SSNMR spectrum for Form I of linerixibat is shown in FIG. 3. Characteristic carbon peaks for Form I include: 161.0, 140.4, 124.7, 65.0, 63.8, 35.8, and 30.4 ppm.

### Example 1f—Single Crystal Structure of Form I

The measured single crystal of the free base linerixibat neat form was prepared by slow cooling from a mixed acetonitrile/water solution.

Single crystal data were collected on a Bruker D8 Venture system using an Incoatec microfocus 3.0 CuKα Source. Data collection and unit cell Indexing were performed in the APEX3 v2017.3-0 suite (Bruker AXS Inc., 2017); processing of the measured intensity data was carried out with the SAINT V8.38A software package (Bruker AXS Inc., 2017). The structures were solved by direct methods using the SHELXT-2018/2 software package (Sheldrick, 2018). The derived atomic parameters (coordinates and temperature factors) were refined through full matrix least-squares in SHELXL-2018/3 (Sheldrick, 2018). Hydrogens were introduced in idealized positions.

Single crystal X-ray data were measured at low temperature (−123° C.). The single crystal was confirmed as linerixibat structure with the following unit cell parameters:
a=9.0428(5) Å; b=18.5813(9) Å; c=35.320(2) Å; α=β=γ=90°;
V 5934.7(6) Å3;
Space group P2₁2₁2₁;
Drug molecules/unit cell 8;
Z′=2 wherein Z′ is the number of drug molecules per asymmetric unit;
Density (calculated) 1.224 g/cm³.

### Example 2

### Form III of Linerixibat

### Example 2a—Preparation Method of Form III

### Method 1

A slurry of Form I of linerixibat (4.93 g) in a mixture of isopropyl alcohol (IPA)/water (7:3 v/v, 70 mL) was prepared and heated to 40° C., seeded with Form III (20 mg) slurried in IPA/water (7:3 v/v, 0.5 mL), then temperature cycled between 40-0° C. for 2 days. The slurry was filtered under vacuum and washed with IPA/water (7:3 v/v, 5 mL). The solid was dried in a vacuum oven at 50° C. for 2 days. The dried product (4.559 g) was analysed by XRPD and confirmed to be Form III GSK2330672B.

### Method 2

96.6 g (1 wt) of GSK2330672B was suspended in 1353 mL (14 vol) of 7:3 isopropyl alcohol(IPA)/water. The slurry was then agitated and thermocycled for 76 h as follows: the slurry was heated to 40° C., over approximately 1 h, held for approximately 1 h, cooled to 0° C. over approximately 1 h and held at 0° C. for approximately 1 h. The product, once at 0° C. for at least 1 h, was then filtered and washed with 96.6 mL (1 vol) of 7:3 IPA/water chilled to 2-8° C. and blown down for 1 h. The resulting solids were then agitator-dried at 50° C. under vacuum to give GSK2330672B Form III solid.

### Method 3

A reactor (Reactor 1) was charged with 6.00 kg GSK2330672B followed by isopropyl alcohol (IPA) (9.8 vols) and purified water (4.2 vols). The following temperature cycle was performed three times: heated to 35-45° C. for 1-3 hrs and held for 1-2 hrs, then cooled to −2-6° C. for 1-3 hrs and held for 1-2 hrs. The suspension was filtered and washed with IPA/water (2.6:1.2 v/v, 3.8 vols). The filter cake was blown down with nitrogen, dried at 15-25° C. under vacuum without agitation for 12 hrs, dried at 45-55° C. under vacuum without agitation for 3 hrs, then dried at 45-55° C. under vacuum with intermittent agitation until residual IPA was no greater than 0.5 (% w/w) by gas chromatography (GC). The dried solid was sieved with 20 mesh sieve size to give 5.60 kg Form III GSK2330672B.

### Example 2b—XRPD of Form III

The X-ray powder diffraction (XRPD) pattern of Form III of linerixibat is shown in FIG. 4 and a summary of the diffraction angle and d-spacings is given in Table 2 below. The XRPD data were acquired using the same apparatus and conditions as Example 1c.

TABLE 2

| Peak No. | Diffraction Angles, 2θ (°) | d-spacings (Å) |
|---|---|---|
| 1 | 5.2 | 17.0 |
| 2 | 7.1 | 12.5 |
| 3 | 9.5 | 9.3 |
| 4 | 10.4 | 8.5 |
| 5 | 12.0 | 7.4 |
| 6 | 13.3 | 6.6 |
| 7 | 14.6 | 6.1 |
| 8 | 15.2 | 5.8 |
| 9 | 15.7 | 5.7 |
| 10 | 17.0 | 5.2 |
| 11 | 17.5 | 5.1 |
| 12 | 19.1 | 4.6 |
| 13 | 20.2 | 4.4 |
| 14 | 20.9 | 4.2 |
| 15 | 21.3 | 4.2 |
| 16 | 21.8 | 4.1 |
| 17 | 22.1 | 4.0 |
| 18 | 23.4 | 3.8 |
| 19 | 25.1 | 3.6 |
| 20 | 26.2 | 3.4 |
| 21 | 28.5 | 3.1 |
| 22 | 29.4 | 3.0 |
| 23 | 31.0 | 2.9 |
| 24 | 31.5 | 2.8 |
| 25 | 33.2 | 2.7 |
| 26 | 37.5 | 2.4 |
| 27 | 38.7 | 2.3 |

### Example 2c—DSC of Form III

The DSC thermogram of Form III was obtained using a TA discovery Q2500 and carried out under the same conditions as Example 1d. The DSC thermogram of Form III is shown in FIG. 5 and Form III has a melt onset at approximately 202.6° C. with a peak temperature at about 205.8° C.

### Example 2d—13C Solid State NMR of Form III

The ¹³C SSNMR spectrum for Form III of linerixibat is shown in FIG. 6. The ¹³C SSNMR data were acquired using the same apparatus and conditions as Example 1e. Characteristic carbon peaks for Form III include: 161.6, 145.6, 141.6, 62.7, 34.5, 24.3, 16.7, and 16.0 ppm.

### Example 3

Form Change from Form I to Form III

### Example 3a—Conversion from Form I to Form III

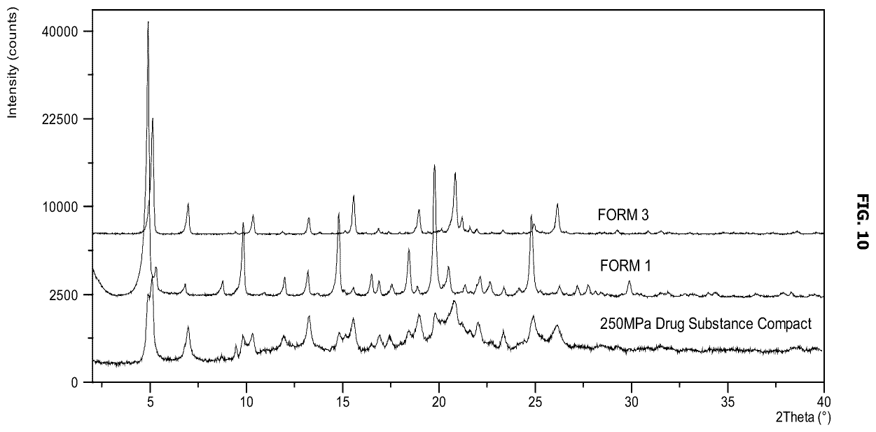

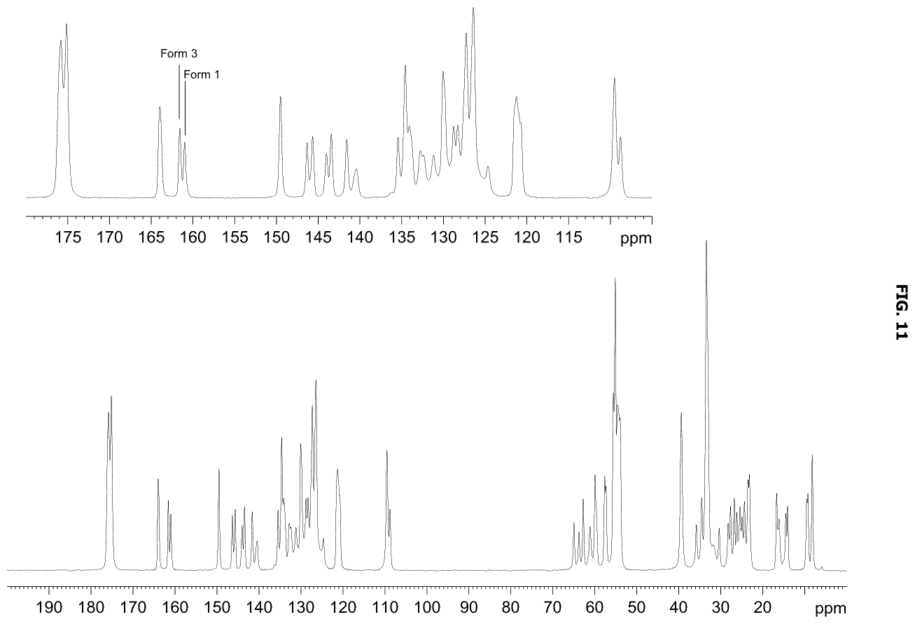

The manufacturing procedures described in Example 1b would generally provide pure Form I. However, the presence of Form III at a low level was observed in one manufacturing batch after the final drying step. Qualitative assessment by ¹³C SSNMR spectroscopy and XRPD showed that Form III content was not greater than about 10% w/w in that batch. Upon investigation, this occurrence was attributed to a combination of a relatively large batch size in a smaller filter drier (agitated drying stage) resulting in the wet material being subjected to greater shear forces during agitation, causing some conversion of Form I to Form III. Conversion of Form I to Form III was also observed during tablet compression process. The level of Form III in the tablet has been observed to have a positive correlation with the applied compression stress used during the formulation process and has been qualitatively estimated at levels up to approximately 40% by weight of the linerixibat drug substance component of the tablet. During formulation development, samples of the linerixibat compression blend were compacted in a device to mimic the full-scale tableting process. X-ray powder diffractograms (XRPD) were obtained on these “compacts.” As the compression force was increased, the XRPD pattern changed to give loss of intensity of some peaks and formation of other peaks. This indicated the presence of another solid state form, which was subsequently confirmed as Form III. FIG. 10 shows an overlay of XRPD patterns of Form I, Form III, and a compact obtained by compressing Form I at 250 MPa. The XRPD data were acquired using the same apparatus and conditions as Example 1c. ¹³C SSNMR spectral analysis was performed on the same compact and it showed the presence of Form III (FIG. 11). Inset of FIG. 11 shows an example of characteristic peaks of Form I and Form III at chemical shifts of 161.0 ppm and 161.6 ppm, respectively. The intensities of Form I and Form III peaks in the ¹³C SSNMR spectrum qualitatively indicate similar populations of the two polymorphic forms in the sample of mixture. The ¹³C SSNMR data were acquired using the same apparatus and conditions as Example 1e.

### Example 3b—The Level of Form III in Tablets

¹³C SSNMR data for linerixibat tablets, 90 mg of Form I compressed with different forces to achieve three different tensile strengths 1.6 MPa, 2.4 MPa and 3.2 MPa, were collected. The ¹³C SSNMR data were acquired using the same apparatus and conditions as Example 1e. A trend of increased Form III content with increasing tensile strength was observed. For the tablets that had a tensile strength within the range investigated, the Form III content was estimated as less than about 40% w/w (as compared to the total weight of linerixibat drug substance component of the tablet).

Process development work indicated that the product manufactured within the tensile strength range investigated will produce a tablet of appropriate physical strength (hardness) across a range of tablet size and shapes. Therefore, for the linerixibat tablets proposed for clinical use, the Form III conversion during compression is estimated to be up to about 40% w/w of the linerixibat drug substance component of the tablet, as the tensile strength is maintained to an acceptable range during compression using tablet core hardness as an in-process control.

### Example 4

### Form II of Linerixibat

### Example 4a—Preparation Method of Form II

### Method 1

Amorphous material prepared by ball milling (1.9 g) and Form II (24.6 mg) were divided equally between four 20-mL vials, and suspended in dichloromethane (6.1 mL) until thickened. Dichloromethane (DCM) (4.1 mL) was added to each vial and the contents stirred for 7 days. Combined slurries were isolated by vacuum filtration on a 0.45 micron filter and deliquored for about an hour to give Form II solid.

### Method 2

To a 125-mL Erlenmeyer flask, a total of 629.1 mg of Form 1 and a total of 112 mL of 3:7 v/v methyl tert-butyl ether/methanol (MTBE/MeOH) and stirred with a stirring bar for 5 hours. The stirring was stopped and the filtered through a WHATMAN 0.45-micron Nylon syringe filter. 53 mL filtered solution was transferred into a clean 120-mL bottle with a loosened cap and the solvent allowed to evaporate for 3 days. The cap was then removed and the bottle was covered with a KIMWIPE and evaporation continued for 9 more days to give 174 mg of Form II solid.

### Example 4b—XRPD of Form II

The X-ray powder diffraction (XRPD) pattern of Form II of linerixibat is shown in FIG. 12 and a summary of the diffraction angle and d-spacings is given in Table 3 below. The XRPD data were acquired using the same apparatus and conditions as Example 1c.

TABLE 3

| Peak No. | Diffraction Angles, °2θ | d-spacings [Å] |
|---|---|---|
| 1 | 5.1 | 17.5 |
| 2 | 6.2 | 14.4 |
| 3 | 7.8 | 11.3 |
| 4 | 10.1 | 8.7 |
| 5 | 11.7 | 7.6 |
| 6 | 13.1 | 6.8 |
| 7 | 14.4 | 6.1 |
| 8 | 15.3 | 5.8 |
| 9 | 16.5 | 5.4 |
| 10 | 17.3 | 5.1 |
| 11 | 18.2 | 4.9 |
| 12 | 19.8 | 4.5 |
| 13 | 20.1 | 4.4 |
| 14 | 20.4 | 4.4 |
| 15 | 20.6 | 4.3 |
| 16 | 21.3 | 4.2 |
| 17 | 22.1 | 4.0 |
| 18 | 23.9 | 3.7 |
| 19 | 24.3 | 3.7 |
| 20 | 25.6 | 3.5 |
| 21 | 27.2 | 3.3 |

### Example 4c—DSC of Form II

The DSC thermogram of Form II was obtained using a TA discovery Q2500 and carried out under the same conditions as Example 1d. The DSC thermogram of Form II is shown in FIG. 13 and Form II has a melt onset at approximately 204.7° C. with a peak temperature at about 206.5° C.

### Example 4d—13C Solid State NMR of Form II

The ¹³C SSNMR spectrum for Form II of linerixibat is shown in FIG. 14. The ¹³C SSNMR data were acquired using a Bruker 400 MHz Avance III HD NMR spectrometer with an operating frequency of 400.22 MHz. The spectrometer was equipped with a 4 mm double resonance magic-angle spinning probe operating at a rotation frequency of 8 kHz. Spectra were obtained using cross-polarisation, with a linear power ramp used on the ¹H channel to enhance cross-polarisation efficiency. Spinning sidebands were eliminated by a total sideband suppression sequence. ¹H decoupling was obtained using the SPINAL-64 sequence. ¹³C chemical shifts are referenced to tetramethylsilane at 0 ppm (parts per million), using the carbonyl peak in α-glycine at 176.4 ppm as a secondary reference.

### Example 5

### Form IV of Linerixibat

### Example 5a—Preparation Method of Form IV

### Method 1

To a 125-mL Erlenmeyer flask, a total of 629.1 mg of Form I and a total of 112 mL of 3:7 v/v methyl tert-butyl ether/methanol (MTBE/MeOH) and stirred with a stirring bar for 5 hours. The stirring was stopped and the filtered through a WHATMAN 0.45-micron Nylon syringe filter. 57 mL of filtered solution was transferred to a 120-mL amber QORPAK bottle and Form IV* seeds (0.54 mg) added. The cap was loosened and slow evaporation started. Two days later, the cap was removed and evaporation continued for a total of 12 days. Wet paste was placed in a vacuum oven with applied vacuum and nitrogen purge and dried overnight at ambient temperature (21.5° C.). Dried material was Form IV. Yield=186 mg.

* Residual Form I present in Form IV seeds.

### Method 2

25 mg GSK2330672B was dispensed into a 2 mL vial and 1.5 mL Methyl tert-butyl ether/methanol (MTBE/MeOH) (3:7 v/v) added and stirred overnight at room temperature to equilibrate. The slurry was filtered through a 0.2 um syringe filter into a clean vial and slowly evaporated with a loosened cap. The slurry was filtered at room temperature and air-dried for 2 hours to give Form IV GSK2330672B.

### Example 5b—XRPD of Form IV

The X-ray powder diffraction (XRPD) pattern of Form IV of linerixibat is shown in FIG. 15 and a summary of the diffraction angle and d-spacings is given in Table 4 below. The XRPD data were acquired using the same apparatus and conditions as Example 1c.

TABLE 4

| Peak No. | Diffraction Angles, °2θ | d-spacings [Å] |
|---|---|---|
| 1 | 5.1 | 17.5 |
| 2 | 10.1 | 8.8 |
| 3 | 12.2 | 7.3 |
| 4 | 15.1 | 5.9 |
| 5 | 20.2 | 4.4 |
| 6 | 25.3 | 3.5 |
| 7 | 30.5 | 2.9 |

### Example 5c—DSC of Form IV

The DSC thermogram of Form IV was obtained using a TA discovery Q2500 and carried out under the same conditions as Example 1d. The DSC thermogram of Form IV is shown in FIG. 16 and Form IV has a melt onset at approximately 195.2° C. with a peak temperature at about 200.4° C.

### Example 5d—13C Solid State NMR of Form IV

The ¹³C SSNMR spectrum for Form IV of linerixibat is shown in FIG. 17. The ¹³C SSNMR data were acquired using a Bruker 400 MHz Avance III HD NMR spectrometer with an operating frequency of 400.22 MHz. The spectrometer was equipped with a 4 mm double resonance magic-angle spinning probe operating at a rotation frequency of 8 kHz. Spectra were obtained using cross-polarisation, with a linear power ramp used on the ¹H channel to enhance cross-polarisation efficiency. Spinning sidebands were eliminated by a total sideband suppression sequence. ¹H decoupling was obtained using the SPINAL-64 sequence. ¹³C chemical shifts are referenced to tetramethylsilane at 0 ppm (parts per million), using the carbonyl peak in α-glycine at 176.4 ppm as a secondary reference.

### Example 6

### Form V of Linerixibat

### Example 6a—Preparation Method of Form V

Amorphous material prepared by ball milling (810.8 mg) was combined with 3:7 v/v ethanol:water (8.0 mL) and a magnetic stir bar in a 20-mL vial. The solvent was added in 1 mL aliquots and shaken with each addition. After adding a total of 4 mL, the suspension was briefly vortexed. The remaining 4 mL were added in 1 mL aliquots and then vortexed again and stirring started room temperature. After 2.5 hours, Form II seeds (3.71 mg, lot 103774-RT-002) were added and the mixture briefly vortexed and stirring was continued. After one day of stirring, the solids were filtered on WHATMAN 1 filter paper with applied vacuum to the Buchner funnel for approximately 1.5 hours. The vacuum was then stopped and the solids covered with a KIMWIPE and left to air dry in a fume hood over a weekend. Vacuum was then restarted to the Buchner funnel and held for 4.5 hours and then the solids were isolated from the filter paper. The isolated yield was 73% (590 mg). XRPD indicated the dried material was Form V.

### Example 6b—XRPD of Form V

The X-ray powder diffraction (XRPD) pattern of Form V of linerixibat is shown in FIG. 18 and a summary of the diffraction angle and d-spacings is given in Table 5 below. The XRPD data were acquired using the same apparatus and conditions as Example 1c.

TABLE 5

| Peak No. | Diffraction Angles, °2θ | d-spacings [Å] |
|---|---|---|
| 1 | 5.3 | 16.8 |
| 2 | 6.2 | 14.3 |
| 3 | 7.1 | 12.5 |
| 4 | 7.8 | 11.4 |
| 5 | 9.5 | 9.3 |
| 6 | 9.8 | 9.0 |
| 7 | 10.2 | 8.6 |
| 8 | 10.5 | 8.4 |
| 9 | 10.7 | 8.2 |
| 10 | 11.7 | 7.6 |
| 11 | 12.2 | 7.3 |
| 12 | 13.1 | 6.8 |
| 13 | 13.5 | 6.5 |
| 14 | 14.7 | 6.0 |
| 15 | 15.2 | 5.8 |
| 16 | 15.8 | 5.6 |
| 17 | 16.8 | 5.3 |
| 18 | 17.2 | 5.2 |
| 19 | 17.5 | 5.1 |
| 20 | 18.3 | 4.9 |
| 21 | 19.0 | 4.7 |
| 22 | 19.5 | 4.5 |
| 23 | 19.7 | 4.5 |
| 24 | 20.3 | 4.4 |
| 25 | 20.5 | 4.3 |
| 26 | 21.1 | 4.2 |
| 27 | 21.6 | 4.1 |
| 28 | 22.5 | 3.9 |
| 29 | 23.1 | 3.8 |
| 30 | 23.9 | 3.7 |
| 31 | 24.4 | 3.6 |
| 32 | 24.8 | 3.6 |
| 33 | 25.6 | 3.5 |
| 34 | 26.5 | 3.4 |
| 35 | 27.8 | 3.2 |
| 36 | 30.4 | 2.9 |
| 37 | 31.0 | 2.9 |
| 38 | 32.4 | 2.8 |
| 39 | 33.0 | 2.7 |
| 40 | 33.8 | 2.6 |
| 41 | 34.1 | 2.6 |
| 42 | 34.6 | 2.6 |
| 43 | 35.1 | 2.6 |
| 44 | 35.7 | 2.5 |
| 45 | 36.9 | 2.4 |
| 46 | 37.7 | 2.4 |
| 47 | 38.6 | 2.3 |
| 48 | 39.3 | 2.3 |
| 49 | 39.6 | 2.3 |

### Example 6c—DSC of Form V

The DSC thermogram of Form V was obtained using a TA discovery Q2500 and carried out under the same conditions as Example 1d. The DSC thermogram of Form V is shown in FIG. 19 and Form V has a melt onset at approximately 197.5° C. with a peak temperature at about 201.3° C.

### Example 6d—13C Solid State NMR of Form V

The ¹³C SSNMR spectrum for Form V of linerixibat is shown in FIG. 20. The ¹³C SSNMR data were acquired using a Bruker 400 MHz Avance III HD NMR spectrometer with an operating frequency of 400.22 MHz. The spectrometer was equipped with a 4 mm double resonance magic-angle spinning probe operating at a rotation frequency of 8 kHz. Spectra were obtained using cross-polarisation, with a linear power ramp used on the ¹H channel to enhance cross-polarisation efficiency. Spinning sidebands were eliminated by a total sideband suppression sequence. ¹H decoupling was obtained using the SPINAL-64 sequence. ¹³C chemical shifts are referenced to tetramethylsilane at 0 ppm (parts per million), using the carbonyl peak in α-glycine at 176.4 ppm as a secondary reference.

### Example 7

### Composition of Linerixibat Tablets, 40 mg

An immediate release tablet formulation for oral administration containing 40 mg of linerixibat (free base) has been developed. Linerixibat Tablets, 40 mg, are manufactured using standard pharmaceutical manufacturing processes (direct compression) and conventional excipients. The tablets are round, purple, and film coated with no markings. The presence of a cosmetic film coat, which will dissolve rapidly in the gastric environment, is expected to have negligible impact on the in vivo performance of the drug, which is locally acting in the distal ileum with a long GI transit time to reach the site of action.

The composition of 40 mg linerixibat tablets is provided in Table C:

TABLE C

| | Quantity | |
|---|---|---|
| Component | (mg/Tablet) | Function |
| Linerixibat¹ | 40.0 | Active |
| Microcrystalline Cellulose¹ | 173.2 | Filler |
| Croscarmellose Sodium | 6.7 | Disintegrant |
| Magnesium Stearate | 2.2 | Lubricant |
| Total Core Tablet (mg) | 222 | — |
| Opadry Purple 03B200014² | 6.7³ | Film Coat |
| Purified Water | qs⁴ | Vehicle |
| Total Film Coated Tablet (mg) | 228.7 | — |

Notes
¹Quantity can be adjusted to reflect the assigned purity of the input drug substance
²Opadry Purple 03B200014 contains Hypromellose (E464) Ph. Eur., or USP, JP, ChP, GB Titanium Dioxide (E171) Ph. Eur. or USP, JP, ChP , GB, FCC, Macrogol/Polyethylene Glycol 400 (E1521) Ph. Eur. or USP/NF, JP, JECFA, FCC, Black iron oxide/Ferrosoferric oxide (E172) NF, JPE, JECFA, ChP, Red Iron oxide (E172) NF, JPE, JECFA, ChP
³The weight of film coat applied per tablet may vary depending on the efficiency of the process but is typically 3% w/w of the tablet core weight
⁴Sufficient quantity to make film coat suspension, then removed during processing

In one embodiment of the invention, there is provided an oral dosage form of linerixibat in a form as disclosed herein (Form I, Form II, Form III, Form IV, Form V, amorphous linerixibat or a mixture of two or more thereof), wherein linerixibat is present in an amount of about 40 mg, and has the composition substantially according to that in Table C.

In another embodiment of the invention, there is provided an oral dosage form of linerixibat which is present as Form I, Form III, or a mixture thereof, wherein linerixibat is present in an amount of about 40 mg, and has the composition substantially according to that in Table C.

### Example 8

### Linerixibat Solubility Profile

### Experiment 8a

The solubility of linerixibat drug substance, in respect of Form I and Form III, was determined over the pH range of 1.2-6.8 at 37±1° C. Eight pH values within this range, including buffers at pH 1.2, 4.5 and 6.8 were evaluated. The therapeutic dose for linerixibat is 40 mg. Therefore, solubility above 0.16 mg/mL, corresponding to a dose of 40 mg being dissolved in 250 mL, is considered high solubility.

The results presented in Tables D and E demonstrate that linerixibat Form I and Form III are both highly soluble at all pHs in the physiological range except close to the solubility minimum (between pH 3.2 and pH 4.2), and that there is no significant difference in solubility between Form I and Form III.

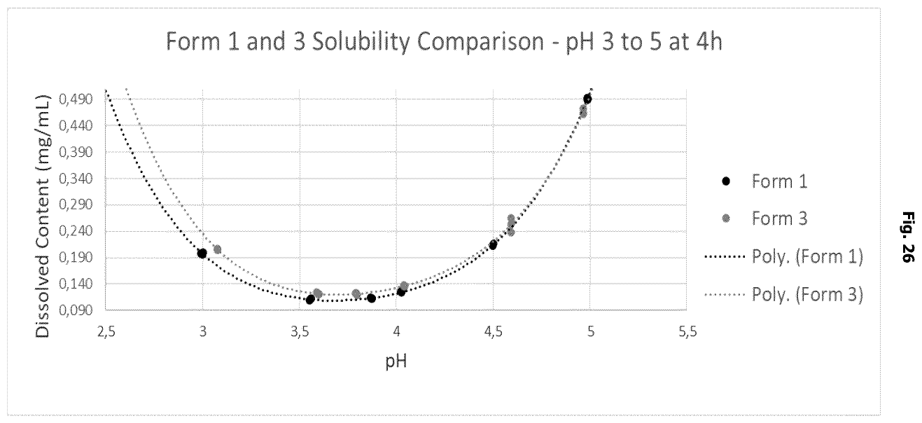

FIG. 26 is a plot of data taken from Tables D and E at 4 h, at a range of between pH3 and pH5.

TABLE D
Solubility Profile of Linerixibat Form I, at 37° C.

| | | 1-hr | | 4-hr | | 24-hr | |
|---|---|---|---|---|---|---|---|
| | Media | timepoint | | timepoint | | timepoint | |
| Nominal | Buffer | Solubility | | Solubility | | Solubility | |
| pH | Type | (mg/mL) | pH | (mg/ml) | pH | (mg/mL) | pH |
| 1.2 | SGF | >1 | 1.2 | >1 | 1.2 | >1 | 1.1 |
| 3.0 | BR | 0.178 | 3.0 | 0.198 | 3.0 | 0.232 | 3.0 |
| 3.5 | BR | 0.099 | 3.6 | 0.110 | 3.6 | 0.128 | 3.6 |
| 3.8 | BR | 0.104 | 3.9 | 0.113 | 3.9 | 0.131 | 3.9 |
| 4.0 | BR | 0.118 | 4.0 | 0.125 | 4.0 | 0.146 | 4.1 |
| 4.5 | Acetate | 0.190 | 4.5 | 0.214 | 4.5 | 0.293 | 4.5 |
| 5.0 | BR | 0.393 | 5.0 | 0.491 | 5.0 | 0.650 | 5.0 |
| 6.8 | Phosphate | >1 | 6.7 | >1 | 6.7 | >1 | 6.7 |

TABLE E
Solubility Profile of Linerixibat Form III, at 37° C.

| | Media | 1-hr timepoint | | 4-hr timepoint | | 24-hr timepoint | |
|---|---|---|---|---|---|---|---|
| Nominal | Buffer | Solubility | | Solubility | | Solubility | |
| pH | Type | (mg/mL) | ph | (mg/ml) | pH | (mg/mL) | pH |
| 1.2 | SGF | >1 | 1.2 | >1 | 1.2 | >1 | 1.1 |
| 3.0 | BR | 0.166 | 3.1 | 0.205 | 3.1 | 0.238 | 3.0 |
| 3.5 | BR | 0.103 | 3.6 | 0.122 | 3.6 | 0.130 | 3.6 |
| 3.7 | BR | 0.104 | 3.8 | 0.121 | 3.8 | 0.129 | 3.8 |
| 4.0 | BR | 0.119 | 4.0 | 0.137 | 4.0 | 0.147 | 4.1 |
| 4.5 | Acetate | 0.199 | 4.6 | 0.252 | 4.6 | 0.310 | 4.6 |
| 5.0 | BR | 0.342 | 5.0 | 0.466 | 5.0 | 0.620 | 5.0 |
| 6.8 | Phosphate | >1 | 6.8 | >1 | 6.7 | >1 | 6.7 |

### Notes for Tables D and E:

SGF=simulated gastric fluid
BR=Britton Robinson

### Experiment 8b

Solubility data was obtained for drug substance samples of linerixibat Form I, linerixibat Form III, and additionally for a sample of Form I compressed to 100 MPa which therefore contained an amount of Form III, in order to replicate typical forces encountered during tablet manufacture.

The media used for the drug substance samples were biorelevant media likely to be experienced in the stomach and the intestine, namely Simulated Gastric Fluid (SGF) over an increasing pH range (1.6, 2.0 and 4.0) and Fasted State Simulated Intestinal Fluid (FaSSIF) at a pH of 6.5. Solubility data was also obtained in 50 mM sodium acetate buffer pH 4.5. The results are shown in Table F.

TABLE F
Solubility Comparison of Linerixibat Form 1 and Form 3 in Various Aqueous Media

| | Solubility as mg/mL | | | | | | | | |
|---|---|---|---|---|---|---|---|---|---|
| | Form | Form | Form 1 | | | Form 1 | Form | Form | Form 1 |
| | 1 | 3 | Compressed | Form 1 | Form 3 | Compressed | 1 | 3 | Compressed |
| | (1 | (1 | to 100 MPa | (4 | (4 | to 100 MPa | (24 | (24 | to 100 MPa |
| Media | Hour) | Hour) | (1 Hour) | Hours) | Hours) | (4 Hours) | Hours) | Hours) | (24 Hours) |
| SGF pH | 7.20 | 7.54 | 7.27 | 7.39 | 7.90 | 7.66 | 7.91 | 8.62 | 8.35 |
| 1.6 | | | | | | | | | |
| SGF pH | 1.49 | 1.49 | 1.56 | 1.53 | 1.58 | 1.62 | 1.62 | 1.70 | 1.70 |
| 2.0 | | | | | | | | | |
| SGF pH | 0.15 | 0.16 | 0.16 | 0.15 | 0.17 | 0.16 | 0.16 | 0.19 | 0.18 |
| 4.0 | | | | | | | | | |
| FaSSIF | 5.08 | 5.2 | 5.25 | 5.11 | 5.31 | 5.45 | 5.31 | 5.32 | 5.49 |
| pH 6.5 | | | | | | | | | |
| Sodium | 0.27 | 0.29 | 0.3 | 0.28 | 0.29 | 0.32 | 0.3 | 0.28 | 0.31 |
| Acetate | | | | | | | | | |
| Buffer | | | | | | | | | |
| pH 4.5 | | | | | | | | | |

### Experiment 8c

Solubility of Form I and Form III was also measured in Britton-Robinson buffers (pH 2-8), determined and compared using a standardised miniaturised shake flask method via an automated platform using a development HPLC method for quantification post centrifugation and filtration. Samples were run at 37° C. and performed as 2 replicate preparations for each pH condition and 4 replicate preparations for each biorelevant media condition at 1, 4 and 24 hours. The results are shown in FIG. 25.

The conclusion based on this data is that the solubility of Form I, Form III and Compressed Form I in biorelevant media is very similar.

### Example 9

### Linerixibat Tablet Dissolution Profile

The dissolution procedure developed for Linerixibat Tablets, 40 mg, is determined as directed in USP<711> and the method parameters are presented in Table G. The percentage of dissolved drug is currently determined by gradient reversed-phase HPLC.

TABLE G

| Dissolution Parameters | Value | |
|---|---|---|
| Apparatus | USP <711> Apparatus II | |
| Sinkers | Three pronged sinkers | |
| Dissolution Media | Potassium dihydrogen phosphate | |
| | buffer, pH 6.8 | |
| Dissolution medium volume | 900 ± 9 | mL |
| Dissolution medium temperature | 37 ± 0.5° | C. |
| Rotation speed | 50 ± 2 | rpm |
| Sampling time | 15 | minutes |

The above dissolution method at pH 6.8 was applied to two batches of Linerixibat Tablets, 40 mg and the profiles obtained are shown in FIG. 24.

It is to be understood that the invention is not limited to the aspects or embodiments illustrated herein above and the right is reserved to the illustrated aspects or embodiments and all modifications coming within the scope of the following claims.

The various references to journals, patents, and other publications which are cited herein comprise the state of the art and are incorporated herein by reference as though fully set forth.

1-34. (canceled)

35. A crystalline form of linerixibat, which is Form III.

36. The crystalline form according to claim 35, wherein Form III is characterized by an X-ray powder diffraction (XRPD) pattern substantially in accordance with FIG. 4.

37. The crystalline form according to claim 35, wherein Form III is characterized by an XRPD pattern comprising at least three or at least four diffraction angles, when measured using Cu K_{α} radiation, selected from the group consisting of about 5.2, 7.1, 10.4, 13.3, 15.7, 19.1, 20.9, and 21.3 degrees 2θ.

38. The crystalline form according to claim 35, wherein Form III is characterized by a ¹³C solid-state NMR (SSNMR) spectrum substantially in accordance with FIG. 6.

39. A mixture of i) the crystalline Form III of linerixibat according to claim 35, and ii) crystalline Form I of linerixibat.

40. The mixture according to claim 39, wherein the crystalline Form III is characterized by (i) an XRPD pattern comprising at least three or at least four diffraction angles, when measured using Cu K_{α} radiation, selected from the group consisting of about 5.2, 7.1, 10.4, 13.3, 15.7, 19.1, 20.9, and 21.3 degrees 2θ, (ii) an X-ray powder diffraction (XRPD) pattern substantially in accordance with FIG. 4, or (iii) a ¹³C solid-state NMR (SSNMR) spectrum substantially in accordance with FIG. 6.

41. The mixture according to claim 39, wherein Form I is characterized by an XRPD pattern substantially in accordance with FIG. 1.

42. The mixture according to claim 39, wherein Form I is characterized by an XRPD pattern comprising at least three or at least four diffraction angles when measured using Cu K_{α} radiation, selected from the group consisting of about 5.0, 5.5, 7.0, 8.9, 9.9, 12.1, 13.3, 14.9, 18.6, 19.9, 20.6, and 22.3 degrees 2θ.

43. The mixture according to claim 39, wherein Form I is characterized by a ¹³C SSNMR spectrum substantially in accordance with FIG. 3.

44. A crystalline form of linerixibat, which is Form II.

45. The crystalline form according to claim 44, wherein Form II is characterized by an X-ray powder diffraction (XRPD) pattern substantially in accordance with FIG. 12.

46. The crystalline form according to claim 44, wherein Form II is characterized by an XRPD pattern comprising at least three or at least four diffraction angles, when measured using Cu K_{α} radiation, selected from the group consisting of about 6.2, 7.8, 10.1, 13.1, 14.4 and 17.3 degrees 2θ.

47. The crystalline form according to claim 44, wherein Form II is characterized by a ¹³C solid-state NMR (SSNMR) spectrum substantially in accordance with FIG. 14.

48. A crystalline form of linerixibat, which is Form IV.

49. The crystalline form according to claim 48, wherein Form IV is characterized by an X-ray powder diffraction (XRPD) pattern substantially in accordance with FIG. 15.

50. The crystalline form according to claim 48, wherein Form IV is characterized by an XRPD pattern comprising at least three, at least four, at least five, at least six, or at least seven diffraction angles, when measured using Cu K_{α} radiation, selected from the group consisting of about 5.1, 10.1, 12.2, 15.1, 20.2, 25.3 and 30.5 degrees 2θ.

51. The crystalline form according to claim 48, wherein Form IV is characterized by a ¹³C solid-state NMR (SSNMR) spectrum substantially in accordance with FIG. 17.

52. A crystalline form of linerixibat, which is Form V.

53. The crystalline form according to claim 52, wherein Form V is characterized by an X-ray powder diffraction (XRPD) pattern substantially in accordance with FIG. 18.

54. The crystalline form according to claim 52, wherein Form V is characterized by an XRPD pattern comprising at least three, at least four, at least five, at least six, or at least seven diffraction angles, when measured using Cu K_{α} radiation, selected from the group consisting of about 5.3, 7.1, 9.5, 10.7, 12.2, 15.2, 15.8, 17.2, 17.5, 19.0, 19.5, 19.7, 20.3, 20.5, 21.1, 21.6, 23.9, 24.4, 24.8, 25.6 and 26.5 degrees 2θ.

55. The crystalline form according to claim 52, wherein Form V is characterized by a ¹³C solid-state NMR (SSNMR) spectrum substantially in accordance with FIG. 20.

56. An amorphous form of linerixibat.

57. A composition comprising linerixibat in a form which is Form II, Form III, Form IV, Form V or an amorphous form, or a mixture of two or more thereof, the mixture optionally comprising Form I of linerixibat.

58. The composition according to claim 57, wherein the composition comprises Form III linerixibat.

59. The composition according to claim 57, wherein the composition comprises a mixture of crystalline Form I and III of linerixibat.

60. The composition according to claim 59, wherein Form III is present in an amount of about 10% to 40% by weight of the linerixibat drug substance component of the composition.

61. The composition according to claim 57, wherein linerixibat is present in an amount of about 40 mg.

62. A pharmaceutical composition comprising the composition according to claim 57, further comprising a pharmaceutically acceptable excipient.

63. The pharmaceutical composition according to claim 62, wherein the pharmaceutical composition is for oral administration.

64. The pharmaceutical composition according to claim 63, wherein the pharmaceutical composition is a tablet or a capsule.

65. The pharmaceutical composition according to claim 64, wherein the pharmaceutical composition is a tablet.

66. A method of treating cholestatic pruritus in a patient with primary biliary cholangitis comprising administering to the patient an effective amount of the pharmaceutical composition according to claim 62.

67. A method of treating cholestatic pruritus in a patient with primary biliary cholangitis comprising administering to the patient an effective amount of the pharmaceutical composition according to claim 63.

68. A method of treating cholestatic pruritus in a patient with primary biliary cholangitis comprising administering to the patient an effective amount of the pharmaceutical composition according to claim 64.

69. A method of treating cholestatic pruritus in a patient with primary biliary cholangitis comprising administering to the patient an effective amount of the pharmaceutical composition according to claim 65.
